(19) United States
(12) Patent Application Publication
(10) Pub. No.: US 2014/0088021 A1
(43) Pub. Date: Mar. 27, 2014

(54) Water-Soluble Polymer-Linked Binding Moiety and Drug Compounds

(71) Applicants: Jennifer Riggs-Sauthier, San Francisco, CA (US); Deborah H. Charych, Albany, CA (US); Clark Norman Eid, Madison, AL (US); Dennis G. Fry, Pacifica, CA (US); Marina Konakova, San Francisco, CA (US); Christine Frances Loehrlein, Alameda, CA (US)

(72) Inventors: Jennifer Riggs-Sauthier, San Francisco, CA (US); Deborah H. Charych, Albany, CA (US); Clark Norman Eid, Madison, AL (US); Dennis G. Fry, Pacifica, CA (US); Marina Konakova, San Francisco, CA (US); Christine Frances Loehrlein, Alameda, CA (US)

(73) Assignee: Nektar Therapeutics, San Francisco, CA (US)

(21) Appl. No.: 14/122,384

(22) PCT Filed: May 24, 2012
(86) PCT No.: PCT/US2012/039453

§ 371 Date: Nov. 26, 2013

Related U.S. Application Data
(60) Provisional application No. 61/491,133, filed on May 27, 2011.

(51) Int. Cl. A61K 47/48

(52) U.S. Cl. CPC A61K 47/48215
USPC 514/19.3; 530/408; 514/21.2

(57) ABSTRACT

Compounds comprising a binding moiety, water-soluble, non-peptidic polymer, and drug are provided. Also provided are methods preparing such compounds, compositions comprising such compounds and methods for administering and using such compounds and compositions.

This application claims the benefit of priority under 35 U.S.C. §119(e) to U.S. Provisional Patent Application Ser. No. 61/491,133 filed on May 27, 2011, the disclosure of which is incorporated herein by reference.

The present invention relates generally to biologically active compounds. In addition, the invention relates to methods for preparing the compounds, pharmaceutical preparations comprising the compounds, and the like.

Human Epidermal Growth Factor Receptor 2 (HER2) is a transmembrane receptor that is highly expressed in a significant number of cancers, including breast cancer, ovarian cancer, and gastric cancer. Overexpression of HER2 is present in about twenty-five percent of all breast cancer and is associated with malignant transformation of cells. HER2 positive breast cancer is generally much more aggressive and has a diminished survival rate compared to breast cancer without HER2 overexpression. Additionally, overexpression of HER2 in gastric cancer is associated with a poor prognosis.

With HER2 overexpression associated with aggressive forms of cancer, there has been an effort to develop therapies that work through the HER2 receptor. Trastuzamab (Herceptin®) is one example that has demonstrated significant success in treating HER2 positive cancers. A humanized monoclonal antibody, trastuzamab targets the extracellular domain of HER2. While effective in some patients, not all patients respond to trastuzamab therapy. Further, some cells develop resistance to monotherapy, requiring combination with other anticancer drugs, such as docetaxel, paclitaxel, anthracylcin, and lapatinib.

Alternatively, given the implications of the overexpression of HER2, the HER2 receptor is an attractive target for directed drug delivery systems. Since there is no natural ligand for HER2, artificial ligands have been developed for targeted drug delivery to the HER2 receptor. These artificial ligands include antibody, Fab, ScFv, peptide, and Affibody® molecules that target the HER2 receptor. These molecules have been used as drug targeting ligands with varying degrees of success.

For example, Affibody® (Affibody AB, Stockholm Sweden) molecules are small (˜0.6 kD), stable proteins based on a 58 amino acid chain of protein A. The binding pockets of an Affibody®, which consist of 13 amino acids, may be optimized to bind to particular targets, one of which is the HER2 receptor. Affibody® molecules offer advantages over other ligands, such as antibodies, due to their small size, ease of synthesis, stability, and reversible folding characteristics. Further, they have been shown to have high affinity for their targets. Alexis et al., (2008) Chem. Med. Chem., vol. 3, pp. 1839-43. With respect to HER2, Affibodys have been shown to selectively bind to HER2 overexpressing cell lines and HER2 specific Affibodys have been reportedly used to direct nanoparticles containing anti-cancer agents to HER2 overexpressing cells. Alexis et al. Attempts to use the affinity of Affibody® molecules as targeting agents has been reported. U.S. Patent Application Publication 2009/0074828.

Beyond directing molecules to cells, conjugates of a targeting moiety and an active agent are believed to produce additional benefits. It has been hypothesized that by increasing delivery of an active agent to a particular cell while reducing normal tissue exposure, the therapeutic window of the active agent may be expanded. In such cases, the maximum tolerated dose may be increased, while the minimum effect dose is decreased. As such, it may be possible to administer highly cytotoxic drugs that normally are avoided due to a narrow therapeutic window. As an example, T-DM1 is an antibody-drug conjugate currently being developed by Immunogen and Genentech. T-DM1 is a conjugate of trastuzamab and DM1 which has reportedly shown efficacy in patients with HER2 metastatic breast cancer. Burris et al., (2011) Journal of Clinical Oncology, pp. 398-405. DM1 is a maytansinoid derivative that is highly potent, but is also considered to be highly toxic. Additionally, antibody-drug conjugates that include a PEG₄Mal linker have been reported. Kovtun et al., (2010), vol. 70, pp 2528-2537.

Although showing promise, there remains a need to (among other things) provide for targeted delivery of a drug, toxin or radioactive substance to HER2 overexpressing cells and other cell targets. The present invention is believed to address these and other needs in the art.

Accordingly, in one or more embodiments of the invention, the invention provides a compound comprising a water-soluble, non-peptidic polymer to which is attached: (a) one or more binding moieties, each attached either directly or via a spacer moiety of one or more atoms; and (b) one or more pharmacologically active agents, each attached either directly or via a spacer moiety of one or more atoms.

In one or more embodiments, a compound of Formula I is provided

Ab-X¹-POLY-X²-Dr (Formula I)

wherein: Ab is a binding moiety; X¹ is a first spacer moiety; POLY is a water-soluble, non-peptidic polymer; X² is a second spacer moiety; and Dr is a pharmacologically active agent.

In one or more embodiments, a compound of Formula II is provided:

[Ab-X¹-POLY]ₐ-Q¹-R-Q²-[POLY-X²-Dr]_{b} (Formula II)

wherein: Ab is a binding moiety; X¹ is a first spacer moiety; POLY, for each occurrence, is a water-soluble, non-peptidic polymer; X² is a second spacer moiety; and Dr is a pharmacologically active agent; R is a residue of polyol, polythiol or polyamine bearing at from 3 to about 50 hydroxyl, thiol or amino groups; Q¹ and Q² for each occurrence is a linker; (a) is an integer from 1 to 49; and (b) is an integer from 1 to 49; provided that (a)+(b) is not less than 3 and not greater than 50.

In one or more embodiments, a composition is provided, the composition comprising a compound comprising a water-soluble, non-peptidic polymer to which is attached: (a) one or more binding moieties, each attached either directly or via a spacer moiety of one or more atoms; and (b) one or more pharmacologically active agents, each attached either directly or via a spacer moiety of one or more atoms.

In one or more embodiments, a pharmaceutical composition is provided, the pharmaceutical composition comprising: (i) a compound comprising a water-soluble, non-peptidic polymer to which is attached: (a) one or more binding moieties, each attached either directly or via a spacer moiety of one or more atoms; and (b) one or more pharmacologically active agents, each attached either directly or via a spacer moiety of one or more atoms; and (ii) a pharmaceutically acceptable excipient.

In one or more embodiments, a composition is provided, the composition comprising a compound of Formula I or Formula II.

In one or more embodiments, a pharmaceutical composition is provided, the pharmaceutical composition comprising: (i) compound of Formula I or Formula II and (ii) a pharmaceutically acceptable excipient.

In one or more embodiments, a method is provided, the method comprising the comprising the steps of (in any order): covalently attaching a binding moiety to a first terminus of a water-soluble, non-peptidic polymer having a first terminus and a second terminus; and covalently attaching a pharmacologically active agent to the second terminus of the water-soluble, non-peptidic polymer.

In one or more embodiments, a method is provided, the method comprising administering to a patient a compound or composition as described herein.

Additional and novel features of the invention will be set forth in the description that follows, and in part, will become apparent to those skilled in the art upon reading the following, or may be learned by practice of the invention.

### BRIEF DESCRIPTION OF THE DRAWINGS

FIG. 1 is a plot of the signal intensity of IRDye-800 labeled Affibody A in tumor cells SKOV-3 and KB vs. time.

FIG. 2 is a plot of the signal intensity of Cy5.5-20 kD PEG-Affibody A in tumor cells SKOV-3 and KB vs. time.

FIG. 3 is a plot of the ratio of signal intensity in SKOV-3 tumors to normal cells vs. time for the articles tested in Example 3.

FIG. 4 is a plot of the mean RLU vs. concentration for certain compounds of the present invention in a NCI-N87 cell line as more fully described in Example 9.

FIG. 5 is a plot of the mean RLU vs. concentration for certain compounds of the present invention in a SKOV-3 cell line as more fully described in Example 9.

FIG. 6 is a plot of the mean RLU vs. concentration for certain compounds of the present invention in a SK-N-SH cell line as more fully described in Example 9.

FIG. 7 plot of the concentration vs. time for IRDye800-Affibody A, 2 kD PEG-Affibody A IRDye800, 20 kD PEG-Affibody A-IRDye800 and 20 kD PEG₂-Affibody A-IRDye800, as more fully described in Example 10.

FIG. 8 is a plot of mean tumor volumes (in mm³) vs. time, for the experiment more fully described in Example 11.

FIG. 9 is a plot of relative volume (% growth) vs. time, for the experiment more fully described in Example 11.

FIG. 10 is a plot of mean body weight vs. time, for the experiment more fully described in Example 11.

FIG. 11 is a plot of percent body weight gain/loss vs. time, for the experiment more fully described in Example 11.

Before describing further embodiments of the present invention in detail, it is to be understood that this invention is not limited to the particular polymers, synthetic techniques, binding moieties, pharmacologically active agents, and the like, as such may vary.

It must be noted that, as used in this specification and the intended claims, the singular forms “a,” “an,” and “the” include plural referents unless the context clearly dictates otherwise. Thus, for example, reference to “a polymer” includes a single polymer as well as two or more of the same or different polymers, reference to “an optional excipient” refers to a single optional excipient as well as two or more of the same or different optional excipients, and the like. In describing and claiming the present invention, the following terminology will be used in accordance with the definitions described below.

The term “water soluble” as in a “water-soluble polymer” polymer is any polymer that is soluble in water at room temperature. Typically, a water-soluble polymer will transmit at least about 75%, more preferably at least about 95%, of light transmitted by the same solution after filtering. On a weight basis, a water-soluble polymer will, in certain embodiments, be at least about 35% (by weight) soluble in water, in certain embodiments at least about 50% (by weight) soluble in water, in certain embodiments about 70% (by weight) soluble in water, and in certain embodiments, about 85% (by weight) soluble in water. In certain embodiments the water-soluble polymer is about 95% (by weight) soluble in water or completely soluble in water. “Water soluble, non-peptidic polymer” encompasses the above, though with respect to being “non-peptidic,” a polymer is non-peptidic when it has less than 35% (by weight) of amino acid residues.

A “polymer” is a molecule possessing from about 2 to about 4000 or more monomers. Specific polymers for use in the invention include those having a variety of geometries such as linear, branched, or forked, to be described in greater detail below.

The terms “monomer,” “monomeric subunit” and “monomeric unit” are used interchangeably herein and refer to one of the basic structural units of a polymer or polymer. In the case of a homo-polymer, a single repeating structural unit forms the polymer. In the case of a co-polymer, two or more structural units are repeated—either in a pattern or randomly—to form the polymer. In certain embodiments, polymers used in connection with present the invention are homo-polymers. The water-soluble, and the water soluble non-peptidic polymer typically comprises one or more monomers serially attached to form a chain of monomers. The polymer can be formed from a single monomer type (i.e., is homo-polymeric) or two or three monomer types (i.e., is co-polymeric).

“PEG,” “polyethylene glycol” and “poly(ethylene glycol)” as used herein, are interchangeable and encompass any nonpeptidic water-soluble poly(ethylene oxide). Typically, PEGs for use in accordance with the invention comprise the following structure “—(OCH₂CH₂)ₙ—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced, e.g., during a synthetic transformation. Throughout the specification and claims, it should be remembered that the term “PEG” includes structures having various terminal or “end capping” groups and so forth. The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as “branched,” “linear,” “forked,” “multifunctional,” and the like, to be described in greater detail below.

An “end capping group” is generally a non-reactive carbon-containing group attached to a terminal oxygen of a PEG polymer. Exemplary end capping groups comprise a C₁₋₅ alkyl group, such as methyl, ethyl and benzyl), as well as aryl, heteroaryl, cyclo, heterocyclo, and the like. For the purposes of the present invention, certain capping groups have relatively low molecular weights such as methyl or ethyl. The end-capping group can also comprise a detectable label. Such labels include, without limitation, fluorescers, chemiluminescers, moieties used in enzyme labeling, colorimetric labels (e.g., dyes), metal ions, and radioactive moieties.

Molecular weight in the context of a water-soluble polymer or water-soluble, non-peptidic polymer, such as PEG, can be expressed as either a number average molecular weight or a weight average molecular weight. In certain instances, the average molecular weight is recited in Daltons or kilodaltons (kD). When adjacent to a PEG, this value is meant to designate the average molecular weight of the PEG, as opposed to the number of repeating units in the PEG. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation or viscometry to determine weight average molecular weight. The polymers of the invention are typically polydisperse (i.e., number average molecular weight and weight average molecular weight of the polymers are not equal), possessing low polydispersity values of less than about 1.2, in certain embodiments less than about 1.15, in certain embodiments less than about 1.10, in certain embodiments less than about 1.05, and in certain embodiments less than about 1.03.

“Branched”, in reference to the geometry or overall structure of a polymer, refers to a polymer having two or more polymers representing distinct “arms” that extend from a branch point.

“Forked” in reference to the geometry or overall structure of polymer, refers to a polymer having two or more functional groups (typically through one or more atoms) extending from a branch point.

A “branch point” refers to a bifurcation point comprising one or more atoms at which a polymer branches or forks from a linear structure into one or more additional arms.

The terms “active,” “reactive” or “activated” when used in conjunction with a particular functional group, refers to a reactive functional group that reacts readily with an electrophile or a nucleophile on another molecule. This is in contrast to those groups that require strong catalysts or highly impractical reaction conditions in order to react (i.e., a “non-reactive” or “inert” group).

“Not readily reactive,” with reference to a functional group present on a molecule in a reaction mixture, indicates that the group remains largely intact under conditions that are effective to produce a desired reaction in the reaction mixture.

A “protecting group” is a moiety that prevents or blocks reaction of a particular chemically reactive functional group in a molecule under certain reaction conditions. The protecting group will vary depending upon the type of chemically reactive group being protected as well as the reaction conditions to be employed and the presence of additional reactive or protecting groups in the molecule. Functional groups which may be protected include, by way of example, carboxylic acid groups, amino groups, hydroxyl groups, thiol groups, carbonyl groups and the like. Representative protecting groups for carboxylic acids include esters (such as a p-methoxybenzyl ester), amides and hydrazides; for amino groups, carbamates (such as tert-butoxycarbonyl) and amides; for hydroxyl groups, ethers and esters; for thiol groups, thioethers and thioesters; for carbonyl groups, acetals and ketals; and the like. Such protecting groups are well-known to those skilled in the art and are described, for example, in T. W. Greene and G. M. Wuts, Protecting Groups in Organic Synthesis, Third Edition, Wiley, New York, 1999, and references cited therein.

A functional group in “protected form” refers to a functional group bearing a protecting group. As used herein, the term “functional group” or any synonym thereof encompasses protected forms thereof.

The terms “spacer moiety,” “linkage” and “linker” are used herein to refer to a bond or an atom or a collection of atoms optionally used to link interconnecting moieties such as a terminus of a polymer segment and a binding moiety or pharmacologically active agent or an electrophile or nucleophile of a binding moiety or pharmacologically active agent. The spacer moiety may be hydrolytically stable or may include a physiologically hydrolyzable or enzymatically degradable linkage. Unless the context clearly dictates otherwise, a spacer moiety optionally exists between any two elements of a compound (e.g., the provided conjugates comprising a residue of a binding moiety or pharmacologically active agent and water-soluble polymer can attached directly or indirectly through a spacer moiety).

“Electrophile” and “electrophilic group” refer to an ion or atom or collection of atoms, which may be ionic, having an electrophilic center, i.e., a center that is electron seeking, capable of reacting with a nucleophile.

“Nucleophile” and “nucleophilic group” refers to an ion or atom or collection of atoms that may be ionic having a nucleophilic center, i.e., a center that is seeking an electrophilic center or with an electrophile.

A “physiologically cleavable” or “hydrolyzable” or “degradable” bond is a relatively labile bond that reacts with water (i.e., is hydrolyzed) under ordinary physiological conditions. The tendency of a bond to hydrolyze in water under ordinary physiological conditions will depend not only on the general type of linkage connecting two central atoms but also on the substituents attached to these central atoms. Such bonds are generally recognizable by those of ordinary skill in the art. Appropriate hydrolytically unstable or weak linkages include but are not limited to carboxylate ester, phosphate ester, anhydrides, acetals, ketals, acyloxyalkyl ether, imines, orthoesters, peptides, oligonucleotides, thioesters, and carbonates.

An “enzymatically degradable linkage” means a linkage that is subject to degradation by one or more enzymes under ordinary physiological conditions.

A “hydrolytically stable” linkage or bond refers to a chemical moiety or bond, typically a covalent bond, that is substantially stable in water, that is to say, does not undergo hydrolysis under ordinary physiological conditions to any appreciable extent over an extended period of time. Examples of hydrolytically stable linkages include but are not limited to the following: carbon-carbon bonds (e.g., in aliphatic chains), ethers, amides, urethanes, amines, and the like. Generally, a stable linkage is one that exhibits a rate of hydrolysis of less than about 1-2% per day under ordinary physiological conditions. Hydrolysis rates of representative chemical bonds can be found in most standard chemistry textbooks.

“Substantially” or “essentially” means nearly totally or completely, for instance, 95% or greater, in certain embodiments 97% or greater, in certain embodiments 98% or greater, in certain embodiments 99% or greater, in certain embodiments 99.9% or greater, and in certain embodiments 99.99% or greater.

“Monodisperse” refers to a polymer composition wherein substantially all of the polymers in the composition have a well-defined, single molecular weight and defined number of monomers, as determined by chromatography or mass spectrometry. Monodisperse polymer compositions are in one sense pure, that is, substantially comprising molecules having a single and definable number of monomers rather than several different numbers of monomers (i.e., a polymer composition having three or more different polymer sizes). A monodisperse polymer composition possesses a MW/Mn value of 1.0005 or less, and in certain embodiments, a MW/Mn value of 1.0000. By extension, a composition comprised of monodisperse conjugates means that substantially all polymers of all conjugates in the composition have a single and definable number (as a whole number) of monomers rather than a distribution and would possess a MW/Mn value of 1.0005, and in certain embodiments, a MW/Mn value of 1.0000 if the polymer were not attached to the residue of the small molecule drug. A composition comprised of monodisperse conjugates can include, however, one or more nonconjugate substances such as solvents, reagents, excipients, and so forth.

“Bimodal,” in reference to a polymer composition, refers to a polymer composition wherein substantially all polymers in the composition have one of two definable and different numbers (as whole numbers) of monomers rather than a distribution, and whose distribution of molecular weights, when plotted as a number fraction versus molecular weight, appears as two separate identifiable peaks. In certain embodiments, for a bimodal polymer composition as described herein, each peak is generally symmetric about its mean, although the size of the two peaks may differ. Ideally, the polydispersity index of each peak in the bimodal distribution, Mw/Mn, is 1.01 or less, in certain embodiments 1.001 or less, and in certain embodiments 1.0005 or less, and in certain embodiments a MW/Mn value of 1.0000. By extension, a composition comprised of bimodal conjugates means that substantially all polymers of all conjugates in the composition have one of two definable and different numbers (as whole numbers) of monomers rather than a large distribution and would possess a MW/Mn value of 1.01 or less, in certain embodiments 1.001 or less and in certain embodiments 1.0005 or less, and in certain embodiments a MW/Mn value of 1.0000 if the polymer were not attached to the residue of the six-membered heterocyclic nucleoside. A composition comprised of bimodal conjugates can include, however, one or more nonconjugate substances such as solvents, reagents, excipients, and so forth.

The terms “active agent,” “biologically active agent” and “pharmacologically active agent” are used interchangeably herein and are defined to include any agent, drug, compound, composition of matter or mixture that provides some pharmacologic, often beneficial, effect that can be demonstrated in vivo or in vitro. This includes foods, food supplements, nutrients, nutraceuticals, drugs, peptides, vaccines, antibodies, vitamins, and other beneficial agents. As used herein, these terms further include any physiologically or pharmacologically active substance that produces a localized or systemic effect in a patient.

“Alkyl” refers to a hydrocarbon chain, typically ranging from about 1 to 20 atoms in length. Such hydrocarbon chains may be but are not necessarily saturated and may be branched or straight chain, although typically straight chain is preferred. Exemplary alkyl groups include methyl, ethyl, propyl, butyl, pentyl, 1-methylbutyl, 1-ethylpropyl, 3-methylpentyl, and the like. As used herein, “alkyl” includes cycloalkyl when three or more carbon atoms are referenced. An “alkenyl” group is an alkyl of 2 to 20 carbon atoms with at least one carbon-carbon double bond.

The terms “substituted alkyl” or “substituted C_{q-r} alkyl” where q and r are integers identifying the range of carbon atoms contained in the alkyl group, denotes the above alkyl groups that are substituted by one, two or three halo (e.g., F, Cl, Br, I), trifluoromethyl, hydroxy, C₁₋₇ alkyl (e.g., methyl, ethyl, n-propyl, isopropyl, butyl, t-butyl, and so forth), C₁₋₇ alkoxy, C₁₋₇ acyloxy, C₃₋₇ heterocyclic, amino, phenoxy, nitro, carboxy, carboxy, acyl, cyano. The substituted alkyl groups may be substituted once, twice or three times with the same or with different substituents.

“Lower alkyl” refers to an alkyl group containing from 1 to 6 carbon atoms, and may be straight chain or branched, as exemplified by methyl, ethyl, n-butyl, i-butyl, t-butyl. “Lower alkenyl” refers to a lower alkyl group of 2 to 6 carbon atoms having at least one carbon-carbon double bond.

“Alkoxy” refers to an —O—R group, wherein R is alkyl or substituted alkyl, in certain embodiments C₁-C₂₀ alkyl (e.g., methoxy, ethoxy, propyloxy, benzyl, etc.), in certain embodiments C₁-C₇. For the purpose of this invention, alkoxy groups may also include aryl substituents when such substituents follow at least one alkyl group, e.g. benzyloxy. The latter are also called “arylalkoxy” substituents.

“Pharmaceutically acceptable excipient” or “pharmaceutically acceptable carrier” refers to component that can be included in the compositions of the invention in order to provide for a composition that has an advantage (e.g., more suited for administration to a patient) over a composition lacking the component and that is recognized as not causing significant adverse toxicological effects to a patient.

The term “aryl” means an aromatic group having up to 14 carbon atoms. Aryl groups include phenyl, naphthyl, biphenyl, phenanthrenyl, naphthacenyl, and the like. “Substituted phenyl” and “substituted aryl” denote a phenyl group and aryl group, respectively, substituted with one, two, three, four or five (e.g., 1-2, 1-3 or 1-4 substituents) chosen from halo (F, Cl, Br, I), hydroxy, hydroxy, cyano, nitro, alkyl (e.g., C₁₋₆ alkyl), alkoxy (e.g., C₁₋₆ alkoxy), benzyloxy, carboxy, aryl, and so forth.

“Pharmacologically effective amount,” “physiologically effective amount,” and “therapeutically effective amount” are used interchangeably herein to mean the amount of a compound present in a composition that is needed to provide a threshold level of active agent and/or conjugate in the bloodstream or in the target tissue. The precise amount will depend upon numerous factors, e.g., the particular active agent, the components and physical characteristics of the composition, intended patient population, patient considerations, and the like, and can readily be determined by one skilled in the art, based upon the information provided herein and available in the relevant literature.

A “difunctional” polymer is a polymer having two functional groups contained therein, typically at its termini. When the functional groups are the same, the polymer is said to be homodifunctional. When the functional groups are different, the polymer is said to be heterobifunctional.

“Multifunctional” in the context of a polymer of the invention means a polymer having 3 or more functional groups contained therein, where the functional groups may be the same or different. Multifunctional polymers of the invention will typically contain from about 3-100 functional groups, or from 3-50 functional groups, or from 3-25 functional groups, or from 3-15 functional groups, or from 3 to 10 functional groups, or will contain 3, 4, 5, 6, 7, 8, 9 or 10 functional groups within the polymer.

A basic reactant or an acidic reactant described herein include neutral, charged, and any corresponding salt forms thereof which have basic or acidic character under any definition (e.g. Bronsted, Lewis, etc.).

The term “patient” refers to a living organism suffering from or prone to a condition that can be prevented or treated by administration of a conjugate as described herein, typically, but not necessarily, in the form of a water-soluble polymer-small molecule drug conjugate, and includes both humans and animals.

“Optional” or “optionally” means that the subsequently described circumstance may but need not necessarily occur, so that the description includes instances where the circumstance occurs and instances where it does not.

With respect to branched water-soluble, non-peptidic polymers, these polymers typically contain a two discernable end capped water-soluble, non-peptidic polymers connected via a branch point, which is connected through a spacer to either a functional group (prior to conjugation) or small molecule drug residue. Exemplary branched forms of water-soluble, non-peptidic polymers are described herein and in WO 2005/107815, WO 2005/108463, U.S. Pat. Nos. 5,932,462 and 7,026,440, and U.S. Patent Application Publication No. 2005/0009988. Among other benefits, branched water-soluble, non-peptidic polymers—given the presence of two discernable water-soluble, non-peptidic polymers—have the potential to provide greater polymer character compared to, for example, a linear polymer having a single water-soluble, non-peptidic polymer.

As used herein, reference to a “water-soluble, non-peptidic polymer” (e.g., “POLY”) is considered to include branched and multi-arm forms even though two or more discernable water-soluble, non-peptidic polymers can be identified.

With respect to multi-arm water-soluble, non-peptidic polymers, these polymers typically contain three or more discernable water-soluble, non-peptidic polymers, each having the ability to covalently attach to a moiety of interest, and each typically connected to a central core moiety (e.g., a residue of a polyol). Among other benefits, multi-arm water-soluble, non-peptidic polymers—given the ability of each arm to covalently attach to a drug or a binding agent—have the potential to provide greater drug character compared to, for example, a linear polymer having a single drug attached thereto.

In certain embodiments, a compound is provided, the compound comprising a water-soluble, non-peptidic polymer to which is attached: (a) one or more binding moieties, each attached either directly or via a spacer moiety of one or more atoms; and (b) one or more pharmacologically active agents, each attached either directly or via a spacer moiety of one or more atoms. In certain embodiments, the compound is a compound of Formula I

Ab-X¹-POLY-X²-Dr (Formula I)

wherein: Ab is a binding moiety; X¹ is a first spacer moiety; POLY is a water-soluble, non-peptidic polymer; X² is a second spacer moiety; and Dr is a pharmacologically active agent.

In certain embodiments, the compound is a compound of Formula II

[Ab-X¹-POLY]ₐ-Q¹-R-Q²-[POLY-X²-Dr]_{b} (Formula II)

wherein: Ab is a binding moiety; X¹ is a first spacer moiety; POLY, for each occurrence, is a water-soluble, non-peptidic polymer; X² is a second spacer moiety; and Dr is a pharmacologically active agent; R is a residue of polyol, polythiol or polyamine bearing at from 3 to about 50 hydroxyl, thiol or amino groups; Q¹ and Q² for each occurrence is a linker (a) is an integer from 1 to 49; and (b) is an integer from 1 to 49; provided that (a)+(b) is not less than 3 and not greater than 50.

In certain embodiments, (a)+(b) is not less than 3 and not greater than 10. In certain embodiments, (a)+(b) is selected from 3, 4, 5, 6, 7, or 8.

In certain embodiments, Ab is an Affibody® moiety. In certain embodiments, Ab is a HER2 binding Affibody®. In certain embodiments, Ab is a carbonic anhydrase 1× binding Affibody®.

In certain embodiments, X¹ is physiologically cleavable. In certain embodiments X² is physiologically cleavable.

The compound of any one of the proceeding claims wherein, X¹ is not physiologically cleavable. In certain embodiments, X² is not physiologically cleavable.

In certain embodiments, Dr is a pharmacologically active agent useful for the treatment of breast cancer, ovarian cancer, pancreatic cancer, stomach cancer, salivary cancer, lung cancer, and esophageal cancer. In certain embodiments Dr is selected from topotecan, docetaxel, paclitaxel, anthracylcin, DM1, and lapatinib. In certain embodiments, Dr is docetaxel.

In certain embodiments, the compound is a compound of the formula

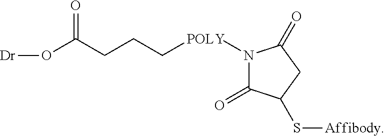

In certain embodiments, each POLY is selected from the group consisting of poly(alkylene oxide), poly(vinyl pyrrolidone), poly(vinyl alcohol), polyoxazoline, and poly(acryloylmorpholine). In certain embodiments POLY is a poly(alkylene oxide). In certain embodiments, POLY is poly(ethylene glycol). In certain embodiments the weight-average molecular weight of the water-soluble non-peptidic polymer in the compound is from about 100 Daltons to about 150,000 Daltons. In certain embodiments, the weight-average molecular weight of the water-soluble non-peptidic polymer in the compound is from about 2,000 to about 25,000. In certain embodiments, the weight-average molecular weight of the water-soluble non-peptidic polymer in the compound is from about 4,000 to about 20,000.

In certain embodiments, the compound is selected from a compound of the formula

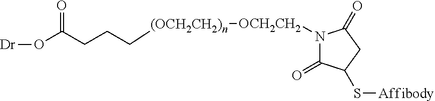

wherein Dr is as defined herein and n is an integer from 2 to 4000. In certain embodiments, n is an integer from 2 to 2000. In certain embodiments, n is an integer from 2 to 1000. In certain embodiments, n is an integer from 20 to 1000. In certain embodiments, wherein n is an integer from 30 to 500. In certain embodiments, n is an integer from 45 to 460.

While not being bound to any one theory, it is believed that the compounds disclosed herein will provide for the targeted delivery of pharmacologically active agents. The compounds are expected to have a longer half-life than the parent binding moiety. Further, by nature of the targeted delivery, it may be possible to lower the dose of the pharmacologically active agent administered to the patient, thereby lowering the risk of harmful side effects. Additionally, the compounds may also be able to deliver a larger amount of the pharmacologically active agent to specific targets, thereby providing a larger effective dose to the target. By increasing the dose of drug received at the target site while reducing the amount of drug in non-targeted tissue, the therapeutic window of the pharmacologically active agent may be increased. This effect may allow for the delivery of highly potent pharmacologically active agents whose use is normally limited due to their side effect profiles.

As will be appreciated by one of skill in the art, the various aspects of the presently disclosed compounds may be varied in order to achieve or vary a property of the compounds. Such variables include but are not limited to, for example, the size of the polymer, the shape of the polymer, the affinity of the binding moiety, the activity/potency of the pharmacologically active agent, and the cleavage properties of the linkers. For example, the enhanced permeability and retention effect (EPR effect) that may be seen with compounds targeting cancer cells may be increased or decreased by varying the polymer size and/or architecture. Further, the binding affinity may be modified through the use of different binding moieties.

With respect to the binding moiety, the binding moiety can be an Affibody® that serves to bind to a target or epitope of interest. Those of ordinary skill in the art can identify whether a target or epitope is associated with a useful target in a given patient, and if so, what the target or epitope is and/or how to obtain a binding moiety (e.g., raising an antibody to the epitope) that will bind to that target or epitope. Often, the target will be a xenobiotic, cancer cell, and/or a protein indicative of a diseased state. Other disease states that might be targeted include, for example, inflammation, sites of infection, metabolic diseases, or other diseases where localization of the active drug will improve efficacy and/or safety.

The compounds of the invention, by including a binding moiety, are more likely to localize to the target to which the binding moiety binds. In some instances, the target is located on and/or associated with a larger particle (e.g., a protein, such as a receptor protein, on a cell or virus). In some instances, the target circulates and/or travels in vivo, such as a ligand located on and/or associated with a cell or soluble macromolecule. In some instances, the target may be a receptor or other tissue-specific protein (that is attached to the membrane or part of the extraceullar matrix). Furthermore, because the compounds of the invention also include a pharmacologically active agent, the compounds similarly localize the pharmacologically active agent. In this way, upon administration of a compound of the invention, a pharmacologically active agent can better localize to an area of interest.

For example, binding moieties include Affibody® molecules (Affibody AB, Stockholm, Sweden) (see e.g., U.S. Pat. Nos. 6,955,877, 6,740,734 and 6,602,977, U.S. Patent Publication Nos. US 2010/0048868, US 2011/0020223, and US 2009/0016957, and International Application No. WO 00/63243). Exemplary, yet non-limiting, binding moieties are described below.

Affibody® molecules are small non-immunoglobulin affinity ligands based on a 58-amino-acid Z-domain scaffold, derived from one of the IgG-binding domains of staphylococcal protein A. Orlova, et al., (2007) Cancer Res. 67; 2178. Randomization of 13 amino acid positions in the binding surface of this domain scaffold has been used for construction of combinatorial phagemid libraries, from which Affibody® molecules binding desired target molecules can be selected by phage display. For example, Affibody® molecules have been prepared for binding to HER2, CAIX, PDGFR beta, epidermal growth factor receptor (EGFR, ErbB), prostate specific membrane antigen (PSMA), fibrinogen, human serum albumin (HSA), immunoglobulin A (IgA), immunoglobulin E (IgE), immunoglobulin G (IgG), immunoglobulin M (IgM), interleukin 8 (IL-8), insulin, tumor necrosis factor alpha (TNF-α), transferrin, and transthyretin. Often Affibody® molecules include a C-terminal cysteine which provides a reactive position for chemical modification.

A compound comprising an Affibody® as disclosed herein can be used to direct the compound to any target for which a particular Affibody® binds. A person of skill in the art will readily be able to select the particular Affibody® depending on the result to be achieved. As in certain embodiments of the present invention, the Affibody® selected is one that binds to the HER2 receptor. Such an Affibody® may be selected in order to direct the compounds disclosed herein to cells where the HER2 receptor is overexpressed. Examples of cells that may over express HER2 are known in the art and include breast cancer, ovarian cancer, pancreatic cancer, stomach cancer, salivary cancer, lung cancer, and esophageal cancer. International Patent Application Publication No. WO/2009/080810. Affibodys or other binding moieties can also be directed against targets such as CD33, CD30, carbonic anhydrase IX, IGFR, folate receptor, CD22, 5T-4, CD80, EGFR, CD-19, CTLA-4, Mucin-1, Mucin-16, EpCam, Rank-L, VEGFA, carcinoembryonic antigen-related cell adhesion molecule 5 (CEA), and VEGFR, for example.

The Affibodys® used herein may be prepared using methods known to one of skill in the art. Examples of references that may provide a description of the preparation of Affibodys used herein include, but are not limited to U.S. Pat. Nos. 6,740,734, 6,955,877 and 6,602,977, U.S. Patent Publication Nos. US 2010/0048868, US 2011/0020223, and US 2009/0016957, and International Application No. WO 00/63243. Additionally, Affibodys may be purchased through commercial sources, e.g. Affibody AB (Stockholm, Sweden).

Exemplary, non-limiting targets of the binding moiety are described below.

Carbonic anhydrase IX (CAIX) is a transmembrane protein expressed in greater than 90% of all clear-cell renal cell carcinoma. Bui et al., (2004) Clin. Cancer Res., vol. 9, p. 802. CAIX is viewed as an independent predictor of survival in clear cell renal carcinoma. CAIX expression has also been implicated in other forms of cancer, such as prostate cancer. CAIX is believed to be associated with tumor hypoxia, a condition where tumors lack sufficient oxygen. Swinson et al., (2003) Journal of Clin. Oncology, vol. 21, no. 3, pp. 473-482. Hypoxic tumors may be resistant to chemotherapy and radiotherapy. CAIX has also been implicated in a poor prognosis for non-small cell lung cancer. In instances where a binding moiety targeting CAIX is used as the binding moiety within the compounds of the invention, the overall compound targets CAIX. Thus, when a binding moiety targeting CAIX is used in the compounds of the invention, the pharmacologically active agent may be one known to treat a disease associated with CAIX expression. In certain embodiments, the pharmacologically active agent may be chosen from an agent useful for the treatment of certain cancers, such as for example, clear-cell renal carcinoma.

CD33 (Siglec-3) is a transmembrane receptor and generally understood as a myeloid specific member of the sialic acid-binding receptor family. CD33 is expressed highly on myeloid progenitor cells but at much lower levels in differentiated cells. The CD33 antigen is expressed on a number of hematologic malignancies, including acute myeloid leukemia (AML), chronic myeloid leukemia (CML), myelodysplastic syndrome and several myeloproliferative disorders. In instances where a binding moiety targeting CD33 is used as the binding moiety within the compounds of the invention, the overall compound targets CD33. Thus, when a binding moiety targeting CD33 is used in the compounds of the invention, the pharmacologically active agent may be one known to treat a disease associated with CD33 expression. In certain embodiments, the pharmacologically active agent may be chosen from an agent useful for the treatment of acute myeloid leukemia (AML), chronic myeloid leukemia (CML), and myelodysplastic syndrome.

CD30 is a cell membrane protein of the tumor necrosis factor receptor family and a tumor marker. CD30 is considered a defining marker for Hodgkin's lymphoma as well as other hematological cancers, such as anaplastic large cell lymphoma. Antibody drug conjugates targeting CD30 are currently being developed, including Brentuximab vedotin (Seattle Genetics). In instances where a binding moiety targeting CD30 is used as the binding moiety within the compounds of the invention, the overall compound targets CD30. Thus, when a binding moiety targeting CD30 is used in the compounds of the invention, the pharmacologically active agent may be one known to treat a disease associated with CD30 expression. In certain embodiments, the pharmacologically active agent may be chosen from an agent useful for the treatment of hematological cancers, such as Hodgkin's lymphoma and anaplastic large cell lymphoma.

CD22 is a receptor found on B-cells. It is present in over 90% of B-lymphoid malignancies, making it an attractive target for treating B-cell non-Hodgkin lymphoma (B-NHL). Ogura et al., (2010) Cancer Sci., 101 (8), pp. 1840-5. CD-22 may also be expressed in other B-cell chronic lymphoproliferative disorders (e.g. B cell precursor acute lymphoblastic leukiemia). In instances where a binding moiety targeting CD22 is used as the binding moiety within the compounds of the invention, the overall compound targets CD22. Thus, when a binding moiety targeting CD22 is used in the compounds of the invention, the pharmacologically active agent may be one known to treat a disease associated with CD22 expression. In certain embodiments, the pharmacologically active agent may be chosen from an agent useful for the treatment of certain leukemias and lymphomas, such as B-cell non-Hodgkin lymphoma.

5T4 (Trophoblast glycoprotein) is a transmembrance glycoprotein expressed in various carcinomas. It is associated with various cancers, including gastric, colorectal, pancreatic, renal (e.g. renal cell carcinoma), and non-small cell lung carcinoma. In instances where a binding moiety targeting 5T4 is used as the binding moiety within the compounds of the invention, the overall compound targets 5T4. Thus, when a binding moiety targeting 5T4 is used in the compounds of the invention, the pharmacologically active agent may be one known to treat a disease associated with 5T4 expression. In certain embodiments, the pharmacologically active agent may be chosen from an agent useful for the treatment of certain cancers, including for example, gastric cancer, colorectal cancer, pancreatic cancer, renal (e.g. renal cell carcinoma), and non-small cell lung carcinoma.

Myostatin, also referred to as growth differentiation factor 8, is a member of the TGF-β superfamily of proteins. Myostatin is expressed largely in developing and adult skeletal muscle and functions as a negative regulator of skeletal muscle. Systemic over-expression of myostatin in adult mice leads to muscle wasting [Zimmers et al. (2002) Science 296:1486-1488], while conversely, a myostatin knock-out mouse is characterized by hypertrophy and hyperplasia of the skeletal muscle resulting in two- to three-fold greater muscle mass than their wild type littermates and a decrease in fat accumulation [McPherron et al. (1997) Nature 387:83-90]. A human with a myostatin knock-out mutation was reported to be associated with gross muscle hypertrophy [Scheulke et al. New Eng. J. Med. 350:2682, 2004). Binding moieties (such as the antibody stamulumab) targeting myostatin have been described.

In instances where a binding moiety targeting myostatin is used as the binding moiety within the compounds of the invention, the overall compound targets myostatin. Thus, when an insulin-like growth factor is used as the pharmacologically active agent within the compound, the insulin-like growth factor is localized to myostatin with the expectation that the resulting species is a compound having both mysostatin and insulin-like growth factor moieties, is myogenetic (e.g., when the binding moiety not only binds to mysostatin but also substantially reduces the activity of myostatin upon binding of the binding moiety to myostatin coupled with the presence of the insulin-like growth factor). Moreover, co-localization of myostatin (via a binding moiety) and the insulin-like growth factor moiety within the same molecule may reduce potential off-target effects because insulin-like growth factor produces effects on multiple organ systems.

CD4 is a surface glycoprotein primarily expressed on surface of T lymphocytes, such as helper T cells. It is through the CD4 surface glycoprotein (and a chemokine receptor such as CCR5 or CXCR4) that human immunodeficiency virus (HIV) introduces its viral payload to the T-lymphocyte, ultimately infecting the T-lymphocyte and its ultimate destruction. Binding moieties (such as the antibody ibalizumab) targeting CD4 have been described. For example, anti-CD4 antibodies are described in Reimann et al. (1997) Aids Res. Human Retrovir. 13:933-943, European Patent Nos. EP 0 512 112, 0 840 618, 0 854 885 and 1 266 965, U.S. Pat. Nos. 5,871,732 and 6,136,310, U.S. Patent Application Publication No. US 2006/0051346, and International Patent Application Publication Nos. WO 91/009966, WO 97/46697 and WO 01/43779.

In instances where a binding moiety targeting CD4 is used as the binding moiety within the compounds of the invention, the overall compound targets T-lymphocytes such as helper T cells. Thus, when an anti-HIV drug (e.g., an HIV protease inhibitor, a reverse transcriptase inhibitor, a fusion inhibitor such as enfuvirtide, etc.) is used as the pharmacologically active agent within the compound, the anti-HIV drug is localized to T-lymphocytes. In instances where the anti-HIV drug must pass through the cell membrane and enter the cytoplasm for pharmacologic activity (e.g., HIV protease inhibitor, a reverse transcriptase inhibitor, etc.), it is preferred to have the anti-HIV drug attached via a cleavable linker. In instances where the anti-HIV drug exerts pharmacologic activity at the cell surface (e.g., a fusion inhibitor such as enfuvirtide), it is preferred to have the anti-HIV drug attached via a non-cleavable linker. In addition, in those instances where the binding moiety targeting CD4 also possesses an independent pharmacological effect (e.g., reduces the ability of the HIV to enter the cell, e.g., an HIV-entry inhibitor), the overall compound can have greater anti-HIV activity than would be the case of the same binding moiety targeting CD4 lacking the independent pharmacological effect.

CCR5 or “chemokine (C—C motif) receptor” is a G protein-coupled, seven transmembrane (7-TM) protein expressed on surface of T cells, macrophages and dendritic cells. Entry of HIV into a T cell can be effected through interaction of the CD4 and CCR5 proteins expressed on the T cell. Upon entry into the T cell, HIV infects the T cell and ultimately causes the destruction of the T cell. Binding moieties targeting CCR5 have been described. For example, anti-CCR5 antibodies are described in Bouhlal et al. (2005) J. Immunol. 174:7202-7209.

In instances where a binding moiety targeting CCR5 is used as the binding moiety within the compounds of the invention, the overall compound targets T cells, macrophages and dendritic cells. Thus, when an anti-HIV drug (e.g., an HIV protease inhibitor, a fusion inhibitor such as enfuvirtide, etc.) is used as the pharmacologically active agent within the compound, the anti-HIV drug is localized to T cells. In instances where the anti-HIV drug must pass through the cell membrane and enter the cytoplasm for pharmacologic activity (e.g., HIV protease inhibitor, a reverse transcriptase inhibitor, etc.), it is preferred to have the anti-HIV drug attached via a cleavable linker. In instances where the anti-HIV drug exerts pharmacologic activity at the cell surface (e.g., a fusion inhibitor such as enfuvirtide), it is preferred to have the anti-HIV drug attached via a non-cleavable linker. In addition, in those instances where the binding moiety targeting CCR5 also possesses an independent pharmacological effect (e.g., reduces the ability of the HIV to enter the cell, e.g., and HIV-entry inhibitor), the overall compound can have greater anti-HIV activity than would be the case of the same binding moiety targeting CD4 lacking the independent pharmacological effect.

CXCR4 or “CXC chemokine receptor” is a G protein-coupled, seven transmembrane (7-TM) protein expressed on surface of T cells. Entry of HIV into a T cell can be effected through interaction of the CD4 and CXCR4 proteins expressed on the T cell. Upon entry into the T cell, HIV infects the T cell and ultimately causes the destruction of the T cell. Binding moieties targeting CXCR4 have been described. For example, anti-CXCR4 antibodies are described in Carnec et al. (2005) J. Virology 79:1930-1933.

In instances where a binding moiety targeting CXCR4 is used as the binding moiety within the compounds of the invention, the overall compound targets T cells. Thus, when an anti-HIV drug (e.g., an HIV protease inhibitor, a fusion inhibitor such as enfuvirtide, etc.) is used as the pharmacologically active agent within the compound, the anti-HIV drug is localized to T cells. In instances where the anti-HIV drug must pass through the cell membrane and enter the cytoplasm for pharmacologic activity (e.g., HIV protease inhibitor, a reverse transcriptase inhibitor, etc.), it is preferred to have the anti-HIV drug attached via a cleavable linker. In instances where the anti-HIV drug exerts pharmacologic activity at the cell surface (e.g., a fusion inhibitor such as enfuvirtide), it is preferred to have the anti-HIV drug attached via a non-cleavable linker. In addition, in those instances where the binding moiety targeting CXCR4 also possesses an independent pharmacological effect (e.g., reduces the ability of the HIV to enter the cell, e.g., and HIV-entry inhibitor), the overall compound can have greater anti-HIV activity than would be the case of the same binding moiety targeting CXCR4 lacking the independent pharmacological effect.

Interleukin-1 receptor is a cell surface receptor expressed on the surface of insulin-producing pancreatic beta cells (among other cell types). The endogenous cytokines of interleukin-1 and interleukin-1 receptor antagonist effectively compete to bind to interleukin-1 receptors expressed on pancreatic beta cells; normal and healthy insulin production occurs when interleukin-1 and interleukin-1 receptor antagonists are in the proper balance. Because interleukin-1 receptor antagonist appears to protect against glucose-induced functional impairment and apoptosis of insulin-producing pancreatic beta cells, a decrease in interleukin-1 receptor antagonist can result in the insufficient production of insulin, thereby leading to hyperglycemia and, ultimately, diabetes. Binding moieties targeting interleukin-1 receptor could be useful for incorporation in the described compounds.

In instances where a binding moiety targeting interleukin-1 receptor is used as the binding moiety within the compounds of the invention, the overall compound targets pancreatic beta cells. Thus, when an insulin secretion-promoting drug (e.g., a sulfonylurea such as acetohexamide, chlorpropamide, tolazimide, tolbutamide, gliclazide, glipizide, glyburide and glimeperide) is used as the pharmacologically active agent within the compound, the insulin secretion-promoting drug is localized to insulin-producing beta cells.

Tumor markers include non-endogenous antigenic proteins expressed by cancer cells as well as endogenous proteins over-expressed by cancer cells. Binding moieties (such as the antibody trastuzumab) targeting tumor markers have been described. See Spiridon et al., (200) Clinical Cancer Research 8(6):1720-1730.

In instances where a binding moiety targeting a tumor marker is used as the binding moiety within the compounds of the invention, the overall compound targets cancer cells. Thus, when an anticancer drug (e.g., a taxane) is used as the pharmacologically active agent within the compound, the anticancer drug is localized to a cancer.

Efflux transporters (e.g., PgP, MRP2, and BCRP) are expressed at the apical surface of epithelial cells and brain endothelial cells. These proteins effectively serve as transcompartmental “pumps” to remove foreign or otherwise undesired moieties outside of the compartment into which they passed. Such transporters serve, for example, as a means to eliminate or reduce the ability of pharmacologically active agents to remain in the central nervous system. Binding moieties (such as the antibody UIC2) targeting an efflux transporter have been described.

In instances where a binding moiety targeting an efflux transporter is used as the binding moiety within the compounds of the invention, the overall compound targets efflux transporters, and preferably decreases or eliminates entirely their efflux action. Thus, a pharmacologically active agent within the compound is localized to the efflux transporter. For example, when a pharmacologically active agent is desired to enter and remain in the central nervous system (i.e., a “CNS drug,” such as a neuroleptic, sedative hypnotic, analgesic, antipyschotic, antiviral and cytotoxic), a compound of the invention in which the pharmacologically active agent is a CNS drug and the binding moiety targets an efflux transporter (and preferably decreases or eliminates entirely the efflux action of the efflux transporter), the CNS drug localizes at the efflux transporter. In an embodiment of this particular approach, the linkage near the CNS drug and the remaining compound is cleavable in vivo so as to allow for entry of the CNS drug into the cell.

Produced by plasma cells located in lymph nodes, IgE antibodies represent a class of immunoglobulins involved in the propagation of an allergic response. Following the release of allergen-specific IgE antibodies from the plasma cells upon a first encounter with the allergen, the Fc portion IgE of the IgE antibodies bind to Fc receptors on mast cells. If the allergen comes into contact with the IgE antibody now attached to the mast cell, the mast cells releases histamine and other cytokines, thereby triggering an immune response. Binding moieties targeting Fc receptors or IgE bound to Fe receptors (such as the antibody omalizumab, which has some binding affinity to IgE bound Fc receptors), are known.

In instances where a binding moiety targeting FcεR (a class of receptors associated with mast cells and basophils) is used as the binding moiety within the compounds of the invention, the overall compound targets mast cells and basophils. Thus, when an anti-allergy drug (e.g., cromolyn, antihistamines, pseudoephedrine, etc.) is used as the pharmacologically active agent within the compound, the anti-allergy drug is localized to mast cells and basophils.

Additional exemplary targets of the binding moiety include, without limitation: IGFR, folate receptor alpha, CD80, EGFR, VEGF receptors, CD-19, CTLA-4, Mucin-1, Mucin-16, EpCam, Rank-L (Receptor activator of nuclear factor kappa-B ligand), VEGFA, carcinoembryonic antigen-related cell adhesion moelcule 5 (CEA), CD20, epidermal growth factor-receptor (EGFR) (also referred to as erbB-1 or HER1); erbB-2 (also referred to as HER2); erbB-3 (also referred to as HER3); erbB-4 (also referred to as HER4); frizzled-10 (FZD-10); gp41; gp120; insulin-like growth factor (IGF1); insulin-like growth factor receptor (IGF1R); IGF2R; InsR; mesenchymal-epithelial transition factor receptor (c-MET receptor); outer membrane proteins (Omps) including, for example, OmpA, OmpC, and OmpF (OprF); matrix metalloproteinase-9; matrix metalloproteinase-13; methacillin-resistant S. aureus ABC proteins (MSRA ABC proteins); penicillin-binding proteins (PBPs), including, for example, PBP1, PBP1a, PBP1b, PBP2, PBP2a, PBP2′, PBP3; peptidoglycan (also referred to as murein); prostate-specific antigen (PSA); prostate-specific membrane antigen (PSMA); pneumococcal surface adhesion protein A (PsaA); receptor activator for nuclear factor κ B (RANK); receptor activator for nuclear factor κ B ligand (RANKL); sclerostin; S. pneumoniae Pit1 (also referred to as Piu); S. pneumoniae Pit2 (also referred to as Pia); uPA; uPAR; and vancomycin-resistant E. faecium ABC proteins. As recited above, when the binding moiety within the compounds of the present invention targets one of the above receptor/protein, the overall compound targets that receptor/protein. In certain embodiments, the pharmacologically active agent will be selected from an agent that is known to treat the disease or diseases associated with the expression of the particular target receptor/protein.

With respect to the pharmacologically active agent, any active agent can be used and the invention is not limited in this regard. Suitable agents can be selected from, for example, hypnotics and sedatives, psychic energizers, tranquilizers, respiratory drugs, anticonvulsants, muscle relaxants, antiparkinson agents (e.g., dopamine antagnonists), analgesics, anti-inflammatories, antianxiety drugs (e.g., anxiolytics), appetite suppressants, antimigraine agents, muscle contractants, anti-infectives (e.g., antibiotics, antivirals, antifungals and vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents (including contraceptives), sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, antibodies, diagnostic agents, and contrasting agents.

The pharmacologically active agent can be any agent that has pharmacological activity and includes (without limitation) proteins, peptides, polypeptides, small molecule drugs, nucleic acids, oligonucleic acids, RNA, DNA, toxins and radioactive substances. In one or more embodiments, the pharmacologically active agent is selected from the group consisting of small molecule drugs, toxins and radioactive substances.

The agent selected from the group consisting of small molecule drugs, toxins and radioactive substances can be any such agent and the invention is not limited in this regard. Typically, the agent so selected is one that will bring therapeutic or clinical value to a patient, particularly in the context of believing delivered or targeted to a specific area or substance in the body.

Exemplary agents selected from the group consisting of small molecule drugs, toxins and radioactive substances include, without limitation, those falling into the class of respiratory drugs, anticonvulsants, muscle relaxants, anti-inflammatories, appetite suppressants, antimigraine agents, muscle contractants, anti-infectives (antibiotics, antivirals, antifungals, vaccines) antiarthritics, antimalarials, antiemetics, bronchodilators, antithrombotic agents, antihypertensives, cardiovascular drugs, antiarrhythmics, antioxicants, anti-asthma agents, diuretics, lipid regulating agents, antiandrogenic agents, antiparasitics, anticoagulants, neoplastics, antineoplastics, hypoglycemics, nutritional agents and supplements, growth supplements, antienteritis agents, immunosupprressants, diagnostic agents, and contrasting agents.

Exemplary agents selected from the group consisting of small molecule drugs, toxins and radioactive substances include, without limitation, adefovir, alosetron, amifostine, amiodarone, aminocaproic acid, aminohippurate sodium, aminoglutethimide, aminolevulinic acid, aminosalicylic acid, amsacrine, anagrelide, anastrozole, aripiprazole, asparaginase, anthracyclines, bexarotene, bicalutamide, bleomycin, BMS-378806 (BMS-806), bortezomib, buserelin, busulfan, cabergoline, capecitabine, carboplatin, carmustine, chlorambucin, celgosir, cilastatin, cisplatin, cladribine, clodronate, cyclophosphamide, cyproterone, cytarabine, camptothecins, 13-cis retinoic acid, all trans retinoic acid; dacarbazine, dactinomycin, daunorubicin, deferoxamine, dexamethasone, diclofenac, diethylstilbestrol, docetaxel, doxorubicin, dutasteride, enfuvirtide, epirubicin, estramustine, etoposide, exemestane, ezetimibe, fexofenadine, fludarabine, fludrocortisone, fluorouracil, fluoxymesterone, flutamide, fondaparinux, fulvestrant, gamma-hydroxybutyrate, gemcitabine, epinephrine, L-Dopa, hydroxyurea, idarubicin, ifosfamide, imatinib, irinotecan, itraconazole, goserelin, lapatinib, letrozole, leucovorin, levamisole, lisinopril, lovothyroxine sodium, lomustine, mechlorethamine, medroxyprogesterone, megestrol, melphalan, mercaptopurine, metaraminol bitartrate, methotrexate, metoclopramide, mexiletine, mitomycin, mitotane, mitoxantrone, naloxone, nicotine, nilutamide, nitisinone, octreotide, oxaliplatin, pamidronate, pentostatin, pilcamycin, porfimer, prednisone, procarbazine, prochlorperazine, ondansetron, palonosetron, oxaliplatin, raltitrexed, ricin, sirolimus, sorafenib, streptozocin, tacrolimus, tandutinib, pimecrolimus, tamoxifen, tegaserod, temozolomide, teniposide, testosterone, tetrahydrocannabinol, thalidomide, thioguanine, thiotepa, topotecan, treprostinil, tretinoin, valdecoxib, celecoxib, rofecoxib, valrubicin, vinblastine, vincristine, vindesine, vinorelbine, voriconazole, dolasetron, granisetron; formoterol, fluticasone, leuprolide, midazolam, alprazolam, amphotericin B, podophylotoxins, nucleoside antivirals, aroyl hydrazones, sumatriptan, eletriptan, macrolides such as erythromycin, oleandomycin, troleandomycin, roxithromycin, clarithromycin, davercin, azithromycin, flurithromycin, dirithromycin, josamycin, spiromycin, midecamycin, loratadine, desloratadine, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin; aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate; polymixins such as polymixin B, capreomycin, bacitracin, penems; penicillins including penicllinase-sensitive agents like penicillin G, penicillin V; penicllinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefmetazole, ceftazidime, loracarbef, and moxalactam, monobactams like aztreonam; and carbapenems such as imipenem, meropenem, and ertapenem, pentamidine isetionate, albuterol sulfate, lidocaine, metaproterenol sulfate, beclomethasone diprepionate, triamcinolone acetamide, budesonide acetonide, fluticasone, ipratropium bromide, flunisolide, cromolyn sodium, and ergotamine tartrate; taxanes such as paclitaxel and cabazitaxel; maytansinoids such as DM1, SN-38, tyrphostines, antihistamines (such as diphenhydramine, bromodiphenhydramine, doxylamine, carbinoxamine, clemastine, dimenhydrinate, tripelennamine, pyrilamine, methapyrilene, thonzylamine, pheniramine, chlorpheniramine, dexchlorpheniramine, bromopheniramine, dexbromopheniramine, pyrrobutamine, triprolidine, promethazine, trimeprazine, methdilazine, cyclizine, chlorcyclizine, diphenylpyraline, phenindamine, dimethindene, meclizine, buclizine, antazoline, cyproheptadine, azatadine, terfenadine, fexofenadine, astemizole, cetirizine, azelastine, azatadine, loratadine, and desloratadine), opiod antagonists (such as naloxone, N-methylnaloxone, 6-amino-14-hydroxy-17-allylnordesomorphine, naltrendol, naltrexone, N-methylnaltrexone, nalbuphine, butorphanol, cyclazocine, pentazocine, nalmephene, naltrendol, naltrindole, nor-binaltorphimine, oxilorphan, 6-amino-6-desoxo-naloxone, pentazocine, levallorphanmethylnaltrexone, buprenorphine, cyclorphan, levalorphan, and nalorphine, as well as those described in U.S. Pat. Nos. 5,159,081, 5,250,542, 5,270,328, and 5,434,171 and in Knapp et a. “The pharmacology of Opiod Peptides” L. F. Tseng Ed., p. 15, Harwood Academic Publishers, 1995), members of the oxymorphone chemical class (including the opioid antagonists above, as well as oxymorphone, codeine, oxycodone, morphine, ethylmorphine, diacetylmorphine, hydromorphone, dihydrocodeine, dihydromorphine, and methyldihydromorphine), platinum coordination complex-based drugs (such as cis-platin, hydroplatin, carboplatin, and oxaliplatin), steroids (such as aldosterone, deoxycorticosterone, fludrocortisone, cortisone, hydrocortisone, prednisolone, prednisone, medrysone, meprednisone, alclometasone, beclomethasone, betamethasone, dexamethasone, diflorasone, flumethasone, methylprednisolone, paramethasone, amcinonide, desonide, fluocinolone, flunisolide, flurandrenolide, triamcinolone, clobetasol, halcinonide, mometasone, clocortolone and desoximetasone), fluoroquinolones (such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin and sitafloxacin), retinoids (such as retinol, retinal, 3-dehydroretinol, α-carotene, β-carotene, γ-carotene, δ-carotene, crytoxanthin, tretinoin, isotretinoin, etretinate, and eretin), phenothiazines (such as phenothiazines, dibenzo-diazepines, galactogugues such as metoclopramide, and thiazides, wherein examples of phenothiazines include prochlorperazine, perphenazine, trifluoroperazine, and fluphenazine, and wherein examples of dibenzo-diazepines include clozapine, olanzapine, and quetiapine, amlodipine, nifedipine, nimodipine, nimodipine, 5-hydroxytryptophan, retinoic acid, nevirapine, and isotretinoin, anticancer drugs and immunosuppressive agents. With regard to anticancer drugs, these include, for example: the alkylating agents, including thiotepa and CTX; alkyl sulfonates including as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide, and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as MTX and 5-fluorouracil (5-FU); folic acid analogues such as denopterin, MTX, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); CTX; thiotepa; taxanes, e.g. paclitaxel (TAXOL®, Bristol-Myers Squibb Oncology, Princeton, N.J.) and docetaxel (TAXOTERE®, Rhone-Poulenc Rorer, Antony, France); chlorambucil; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DFMO); retinoic acid; esperamicins; capecitabine; and pharmaceutically acceptable salts, acids or derivatives of any of the above. With respect to immunosuppressant drugs, these include (without limitation): 2-amino-6-aryl-5-substituted pyrimidines (see U.S. Pat. No. 4,665,077); NSAIDs; ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); bromocryptine; danazol; dapsone; glutaraldehyde; cyclosporin A; dihydrofolate reductase inhibitors such as methotrexate; anti-malarial agents such as chloroquine and hydroxychloroquine; sulfasalazine; leflunomide; cytokine; streptodornase; FK506; RS-61443; chlorambucil; deoxyspergualin; rapamycin; T-cell receptor; T-cell receptor fragments; CTX; chlorambucil; azathioprine, leflunomide, and MMF.

Exemplary active agents that are peptides (or peptide-like moieties) that are suitable for use as a agent include but are not limited to agalsidase, alefacept, aspariginase, amdoxovir (DAPD), antide, becaplermin, calcitonins, cyanovirin, denileukin diftitox, erythropoietin (EPO), EPO agonists (e.g., peptides from about 10-40 amino acids in length and comprising a particular core sequence as described in WO 96/40749), dornase alpha, erythropoiesis stimulating protein (NESP), coagulation factors such as Factor V, Factor VII, Factor VIIa, Factor VIII, Factor IX, Factor X, Factor XII, Factor XIII, von Willebrand factor; ceredase, cerezyme, alpha-glucosidase, collagen, cyclosporin, alpha defensins, beta defensins, desmopressin, exedin-4, granulocyte colony stimulating factor (GCSF), thrombopoietin (TPO), alpha-1 proteinase inhibitor, elcatonin, granulocyte macrophage colony stimulating factor (GMCSF), fibrinogen, filgrastim, growth hormones human growth hormone (hGH), somatropin, growth hormone releasing hormone (GHRH), GRO-beta, GRO-beta antibody, bone morphogenic proteins such as bone morphogenic protein-2, bone morphogenic protein-6, OP-1; acidic fibroblast growth factor, basic fibroblast growth factor, CD-40 ligand, heparin, human serum albumin, low molecular weight heparin (LMWH), interferons such as interferon alpha, interferon beta, interferon gamma, interferon omega, interferon tau, consensus interferon; interleukins and interleukin receptors such as interleukin-1 receptor, interleukin-2, interluekin-2 fusion proteins, interleukin-1 receptor antagonist, interleukin-3, interleukin-4, interleukin-4 receptor, interleukin-6, interleukin-8, interleukin-12, interleukin-13 receptor, interleukin-17 receptor; lactoferrin and lactoferrin fragments, luteinizing hormone releasing hormone (LHRH), insulin, pro-insulin, insulin analogues (e.g., mono-acylated insulin as described in U.S. Pat. No. 5,922,675), amylin, C-peptide, somatostatin, somatostatin analogs including octreotide, vasopressin, follicle stimulating hormone (FSH), influenza vaccine, insulin-like growth factor (IGF), insulintropin, macrophage colony stimulating factor (M-CSF), plasminogen activators such as alteplase, urokinase, reteplase, streptokinase, pamiteplase, lanoteplase, and teneteplase; nerve growth factor (NGF), osteoprotegerin, platelet-derived growth factor, tissue growth factors, transforming growth factor-1, vascular endothelial growth factor, leukemia inhibiting factor, keratinocyte growth factor (KGF), glial growth factor (GGF), T Cell receptors, CD molecules/antigens, tumor necrosis factor (TNF), monocyte chemoattractant protein-1, endothelial growth factors, parathyroid hormone (PTH), glucagon-like peptide, somatotropin, thymosin alpha 1, rasburicase, thymosin alpha 1 IIb/IIIa inhibitor, thymosin beta 10, thymosin beta 9, thymosin beta 4, alpha-1 antitrypsin, phosphodiesterase (PDE) compounds, VLA-4 (very late antigen-4), VLA-4 inhibitors, bisphosphonates, cyanovirin, (CV-N), cyanovirin mutants such as CV-N(Q62C) and others described in U.S. Pat. No. 7,267,941, and cystic fibrosis transmembrane regulator (CFTR) gene, and addition variants, substitution variants, and deletion variants thereof.

In certain embodiments, the pharmacologically active agent is an auristatin, including analogs and derivatives thereof. In certain embodiments the auristatin is auristatin E, monomethyl auristatin E, auristatin PE, and monomethyl auristatin F.

In certain embodiments, the pharmacologically active agent is an epitholone including epitholoines A, B, C, D, E, and F, including analoges and derivatives thereof.

In certain embodiments, the pharmacologically active agent is a maytansinoid. In certain embodiments the pharmacologically active agent is DM1.

One of skill in the art may select the pharmacologically active agent based on the particular binding agent used. For example, in certain embodiments, an Affibody® molecule that targets the HER2 receptor may be used. As such, the pharmacologically active agent may be chosen from one that may treat the underlying condition associated with HER2 overexpression. In certain embodiments the pharmacologically active agent is selected from a compound used for the treatment of breast cancer, ovarian cancer, pancreatic cancer, stomach cancer, salivary cancer, lung cancer, and esophageal cancer. In certain embodiments the pharmacologically active agent is chosen from docetaxel, paclitaxel, anthracylcin, topotecan, gemcitabine and lapatinib.

The above exemplary agents are meant to encompass, where applicable, analogues, agonists, antagonists, inhibitors, isomers, and polymorphs.

With respect to the water-soluble, non-peptidic polymer, any polymer can be used so long as the polymer is non-antigenic and non-immunogenic. In certain embodiments, when the active agents (e.g. proteins) are relatively large, the polymer in the conjugate is water soluble. With respect to relatively small active agents (e.g. nonpeptidic active agents), the polymer in the conjugate can be either water soluble or not. With regard to the polymer associated with conjugates, however, the polymer is typically non-peptidic. In certain embodiments the polymer is a poly(ethylene glycol). In certain embodiments, a polymer suited for use herein can be, for example, other water-soluble poly(alkylene glycols), copolymers of ethylene glycol and propylene glycol and the like, poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline (which are described in WO 2008/106186), poly(N-acryloylmorpholine), such as described in U.S. Pat. No. 5,629,384, and copolymers, terpolymers, nonrandom block, and random block polymers of any of the foregoing.

The water-soluble, non-peptidic polymer is not limited to a particular structure and can be linear (e.g., an end capped, e.g., alkoxy PEG or a bifunctional PEG), branched or multi-armed (e.g., forked PEG or PEG attached to a polyol core), a dendritic (or star) architecture, each with or without one or more linkages. In certain embodiments the linkages may be degradable. Moreover, the internal structure of the water-soluble polymer can be organized in any number of different repeat patterns and can be selected from the group consisting of homopolymer, alternating copolymer, random copolymer, block copolymer, alternating tripolymer, random tripolymer, and block tripolymer. In the context of being present within an overall structure, a water-soluble polymer segment has from 1 to about 300 termini.

In addition to the above-described forms of the polymer, the polymer can also have one or more weak or degradable linkages in the polymer. For example, poly(ethylene glycol) (“PEG”) can be prepared with ester linkages in the polymer that are subject to hydrolysis. As shown below, this hydrolysis results in cleavage of the polymer into fragments of lower molecular weight:

—PEG-CO₂-PEG-+H₂O→-PEG-CO₂H+HO-PEG-

Other hydrolytically degradable linkages, useful as a degradable linkage within the polymer, include carbonate linkages; imine linkages resulting, for example, from reaction of an amine and an aldehyde (see, e.g., Ouchi et al. (1997) Polymer Preprints 38(1):582-3); phosphate ester linkages formed, for example, by reacting an alcohol with a phosphate group; hydrazone linkages which are typically formed by reaction of a hydrazide and an aldehyde; acetal linkages that are typically formed by reaction between an aldehyde and an alcohol; ortho ester linkages that are, for example, formed by reaction between a formate and an alcohol; amide linkages formed by an amine group, e.g., at an end of a polymer such as PEG, and a carboxyl group of another PEG chain; urethane linkages formed from reaction of, e.g., a PEG with a terminal isocyanate group and a PEG alcohol; peptide linkages formed by an amine group, e.g., at an end of a polymer such as PEG, and a carboxyl group of a peptide; and oligonucleotide linkages formed by, for example, a phosphoramidite group, e.g., at the end of a polymer, and a 5′ hydroxyl group of an oligonucleotide.

The polymer is typically biocompatible and non-immunogenic. With respect to biocompatibility, a substance is considered biocompatible if the beneficial effects associated with use of the substance alone or with another substance (e.g., an active agent) in connection with living tissues (e.g., administration to a patient) outweighs any deleterious effects as evaluated by a clinician, e.g., a physician. With respect to non-immunogenicity, a substance is considered non-immunogenic if use of the substance alone or with another substance in connection with living tissues does not produce an immune response (e.g., the formation of antibodies) or, if an immune response is produced, that such a response is not deemed clinically significant or important as evaluated by a clinician.

In certain embodiments, because of its water-solubility, non-immunogenicity and biocompatibility, poly(ethylene glycol) it is a polymer for use in the each of the polymer-active agent conjugate populations of the compositions described herein. Structurally, poly(ethylene glycol) is a linear polymer terminated at each end with hydroxyl groups:

HO—CH₂CH₂O—(CH₂CH₂O)_{m′}—CH₂CH₂—OH

wherein (m′) is a positive integer representing the number of repeating monomers (typically from about 2 to about 4000). Conveniently, poly(ethylene glycol) can be abbreviated as “PEG” and whenever a structure is presented wherein “PEG” appears, it will be assumed that the following structure applies:

—CH₂CH₂O—(CH₂CH₂O)_{m′}—CH₂CH₂

wherein (m′) is as previously defined.

As contemplated by the above structures of Formula II (e.g., [Ab-X¹-POLY]ₐ-Q¹-R-Q²-[POLY-X²-Dr]_{b}), the compound has a number of arms, e.g., from 3 to about 50. An exemplary number of arms includes 3, 4, 5, 6, 7, 8, 9, and 10. In one or more embodiments, the compounds of the invention are prepared from multi-armed polymer reagents, which, in turn, are prepared from multi-arm polymers based on a multi-arm core molecule.

For example, in one approach, a multi-arm polymer can be prepared from a multi-arm core molecule by effectively “growing” a polymer onto each terminus of a multi-arm core molecule. By way of non-limiting example, it is possible to synthesize a polymer arm onto a polyol (e.g., pentaerythritol, diglycerol, etc.) via an ethoxylation reaction. In another exemplary approach, a multi-arm polymer can be prepared from a multi-arm core molecule by attaching a water-soluble, non-peptidic polymer onto each terminus of a multi-arm core molecule. The principles of both approaches are described in the literature and in, for example, U.S. Pat. No. 7,026,440. The invention, however, is not limited with regard to the specific approach taken.

In one or more embodiments, the residue of the polyol, polythiol or polyamine, “R,” used in connection with the multi-arm polymer is an organic radical-containing moiety possessing from about 3 to about 150 carbon atoms (e.g., from about 3 to about 50 carbon atoms, such as 3, 4, 5, 6, 7, 8, 9, and 10). The residue may contain one more heteroatoms (e.g., O, S, or N). In addition, the residue may be linear. In some instances, the residue may be cyclic.

As previously indicated, the residue of the polyol, polythiol or polyamine, “R,” that forms the basis of the branching for the multi-armed compounds provided herein, originated from a corresponding polyol, polythiol or polyamine (prior to be incorporated into the multi-arm structures containing a water-soluble, non-peptidic polymer). In one or more embodiments, the corresponding polyol, polythiol, or a polyamine bears at least three hydroxyl, thiol, or amino groups, respectively, available for polymer attachment. A “polyol” is a molecule comprising three or more hydroxyl groups. A “polythiol” is a molecule that comprises three or more thiol groups. A “polyamine” is a molecule comprising three or more amino groups.

In one or more embodiments, the polyol, polyamine or polythiol will typically contain 3 to about 25 hydroxyl, or amino groups or thiol groups, respectively, such as from 3 to about 10 (i.e., 3, 4, 5, 6, 7, 8, 9, 10) hydroxyl, amino groups or thiol groups, respectively, preferably from 3 to about 8 (i.e., 3, 4, 5, 6, 7, or 8) hydroxyl, amino groups or thiol groups, respectively. In one or more embodiments, the number of atoms between each hydroxyl, thiol, or amino group will vary, although lengths of from about 1 to about 20 (e.g., from 1 to about 5) intervening atoms, such as carbon atoms, between each hydroxyl, thiol or amino group, are exemplary. In referring to intervening core atoms and lengths, —CH₂— is considered as having a length of one intervening atom, —CH₂CH₂— is considered as having a length of two atoms, and so forth.

Exemplary polyols and polyamines (for which corresponding residues could be present in the compounds provided herein) have (Radical)-(OH)ₐ and (Radical)-(NH₂)ₐ structures, respectively, where (Radical) corresponds to an organic-containing radical and q is a positive integer from 3 to about 50. Note that the variable “Q,” when taken together with R, typically represents a residue of the core organic radical as described herein. That is to say, when describing polyols, polythiols and polymer amines, particularly by name, these molecules are being referenced in their form prior to incorporation into a water-soluble polymer-containing structure. So, for example, a compound of Formula II wherein R is a residue of the polyol, pentaerythritol [C(CH₂OH)₄], the residue “R” includes carbon (i.e., “C,”) and together with “Q” represents “C(CH₂O—)₄.”

Illustrative polyols include aliphatic polyols having from 1 to 10 carbon atoms and from 3 to 10 hydroxyl groups, including for example, trihydroxyalkanes, tetrahydroxyalkanes, polyhydroxy alkyl ethers, polyhydroxyalkyl polyethers, and the like. Cycloaliphatic polyols include straight chained or closed-ring sugars and sugar alcohols, such as mannitol, sorbitol, inositol, xylitol, quebrachitol, threitol, arabitol, erythritol, adonitol, dulcitol, facose, ribose, arabinose, xylose, lyxose, rhamnose, galactose, glucose, fructose, sorbose, mannose, pyranose, altrose, talose, tagitose, pyranosides, sucrose, lactose, maltose, and the like. Additional examples of aliphatic polyols include derivatives of glucose, ribose, mannose, galactose, and related stereoisomers. Aromatic polyols may also be used, such as 1,1,1-tris(4′-hydroxyphenyl) alkanes, such as 1,1,1-tris(4-hydroxyphenyl)ethane, 2,6-bis(hydroxyalkyl)cresols, and the like. Other core polyols that may be used include polyhydroxycrown ethers, cyclodextrins, dextrins and other carbohydrates (e.g., monosaccharides, oligosaccharides, and polysaccharides, starches and amylase).

Exemplary polyols include glycerol, trimethylolpropane, pentaerythritol, dipentaerythritol, tripentaerythritol, ethoxylated forms of glycerol, trimethylolpropane, pentaerythritol, dipentaerythritol, tripentaerythritol. Also, preferred are reducing sugars such as sorbitol and glycerol oligomers, such as diglycerol, triglycerol, hexaglycerol and the like. A 21-arm polymer can be synthesized using hydroxypropyl-β-cyclodextrin, which has 21 available hydroxyl groups. Additionally, a polyglycerol having an average of 24 hydroxyl groups is also included as an exemplary polyol.

Exemplary polyamines include aliphatic polyamines such as diethylene triamine, N,N′,N″-trimethyldiethylene triamine, pentamethyl diethylene triamine, triethylene tetramine, tetraethylene pentamine, pentaethylene hexamine, dipropylene triamine, tripropylene tetramine, bis-(3-aminopropyl)-amine, bis-(3-aminopropyl)-methylamine, and N,N-dimethyl-dipropylene-triamine. Naturally occurring polyamines that can be used in the present invention include putrescine, spermidine, and spermine. Numerous suitable pentamines, tetramines, oligoamines, and pentamidine analogs suitable for use in the present invention are described in Bacchi et al. (2002) Antimicrobial Agents and Chemotherapy, 46(1):55-61, which is incorporated by reference herein.

Provided below are illustrative structures corresponding to residues of polyols [although each structure is depicted with the oxygen atom (“O”) derived from the corresponding hydroxyl group, each “O” can be substituted with sulfur (“S”) or NH to depict the corresponding residue of a polythiol or polyamine, respectively). Note that the residues shown below would be understood in terms of compounds of Formula II as corresponding to “R” and “Q.” In any event, conjugates based on any of the illustrative structures set forth below are included as part of the invention.

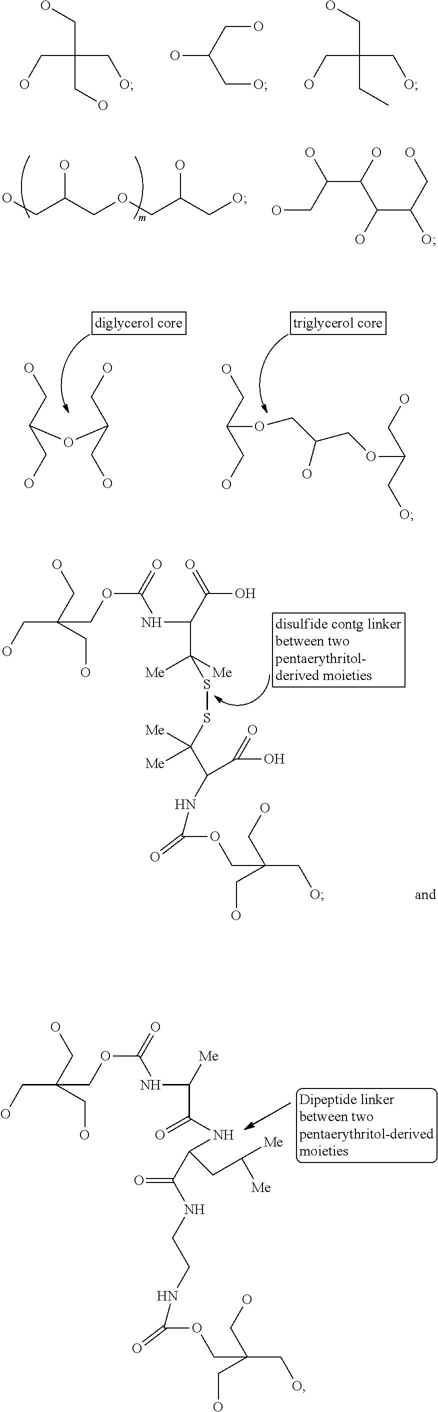

wherein m is a positive integer from 0-40 [e.g., 0-10, for example, 0-5 (i.e., 0, 1, 2, 3, 4, 5)].

Water-soluble, non-peptidic-containing multi-arm polymers (used as, for example, multi-arm polymeric reagents to prepare compounds of the invention) based on the above-described polyols, polythiols and polyamines and others are described in WO 2007/098466, WO 2010/019233 and U.S. Pat. No. 7,744,861. These references and others describe methods for preparing such multi-arm polymers.

The linkers, Q¹ and Q², serve to connect the residue of the polyol, polythiol or polyamine bearing at from 3 to about 50 hydroxyl, thiol or amino groups, “R,” to each water-soluble, non-peptidic polymer, POLY. In this regard, the invention is not limited with respect to the specific linker used. In one or more embodiments, the linker between the residue, “R,” and the water-soluble, non-peptidic polymer, POLY, is a hydrolytically stable linker).

In one or more embodiments of the invention, the linkers, Q¹ and Q², are influenced by the approach used to form the multi-arm polymer employed in preparing the compounds of the invention. For example, if a water-soluble, non-peptidic polymer bearing a functional group reactive to a hydroxyl, thiol or amine is reacted with a polyol, polythiol or polyamine, respectively, the linkers, Q¹ and Q², may include one or more atoms incorporating the bond formed between the termini of the polyol, polythiol or polyamine and the beginning of the repeating monomers of the water-soluble, non-peptidic polymer, POLY. Illustrative linking chemistries in this regard (along with the resulting linkers) are described in the literature and in, for example, Wong (1991) “Chemistry of Protein Conjugation and Crosslinking”, CRC Press, Boca Raton, Fla., and Brinkley (1992) Bioconjug. Chem. 3:2013.

In one or more embodiments of compounds of Formula II, Q¹ and Q², contain at least one heteratom such as O, or S, or NH, where the atom proximal to R in Q¹ and/or Q², when taken together with R, typically represents a residue of an organic radical-containing core of the polyol, polythiol or polyamine. Generally, the linkers, Q¹ and Q², contain from 1 to about 10 atoms (e.g., from 1 to about 5 atoms). The linkers, Q¹ and Q², typically contain a number of atoms selected from the group consisting of: 1, 2, 3, 4, 5, 6, 7, 8, 9, or 10. Illustrative linkers include O, S, —NH—, —NH—C(O)— and —C(O)—NH—.

The remaining variables in Formula II include the water-soluble, non-peptidic polymer, POLY and the releasable linkage-containing spacer moiety, both of which have already been discussed. With respect to compounds encompassed by Formula II, however, typical molecular weights for the water-soluble, non-peptidic polymer (e.g., each POLY) include: about 200, about 250, about 300, about 400, about 500, about 600, about 700, about 800, about 900, about 1,000, about 1,500, about 2,000, about 3,000, about 4,000, about 5,000, about 6,000, about 7,000, about 7,500, about 8,000, about 9,000, about 10,000, about 12,000, about 15,000, about 17,500, about 18,000, about 19,000 and about 20,000 Daltons.

As previously indicated, the compounds described herein include binding moiety and an agent selected from the group consisting of small molecule drugs, toxins and radioactive substances. The conjugates of any given population can be prepared using any technique known to those of ordinary skill in the art. Typically, however, a polymer having at least two different reactive groups (wherein one is optionally blocked) is allowed to react with either the binding moiety or the agent selected from the group consisting of small molecule drugs, toxins and radioactive substances. Thereafter, the optional blocking group of the polymer can be removed using known techniques followed by reaction of the other species (either the antibody moiety or the agent selected from the group consisting of small molecule drugs, toxins and radioactive substances, depending on which species had previously been attached). Conveniently, in the context of linear conjugates, linear, heterobifunctional polymeric reagents (wherein each terminus of the linear polymer has a different reactive group) are known.

Representative polymeric reagents and methods for conjugating these polymeric reagents to an active agent or binding moierty are known in the art and further described in Zalipsky et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992), and in Zalipsky (1995) Advanced Drug Reviews 16:157-182. These reagents may also be available through commercial sources. These and other polymeric reagents are described in more detail below.

The reactive group of the polymeric reagent can be any group suited to react with the binding moiety and the pharmacologically active agent. Generally, the reactive group comprises an electrophilic or nucleophilic group that allows for covalent attachment of the active agent under covalent coupling conditions. Examples of such reactive groups associated with the polymeric reagent include, but are not limited to those selected from the group consisting of hydroxyl (—OH), ester, orthoester, carbonate, acetal, aldehyde, aldehyde hydrate, ketone, ketone hydrate, thione, thione hydrate, hemiketal, sulfur-substituted hemiketal, ketal, alkenyl, acrylate, methacrylate, acrylamide, sulfone, amine, hydrazide, thiol, thiol hydrate, carboxylic acid, isocyanate, isothiocyanate, maleimide

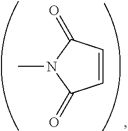

succinimide

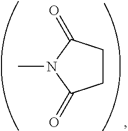

benzotriazole

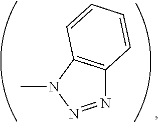

vinylsulfone, chloroethylsulfone, dithiopyridine, vinylpyridine, iodoacetamide, epoxide, glyoxals, diones, mesylates, tosylates, thiosulfonate, tresylate, silane, and protected or activated forms thereof.

In certain embodiments, on either the binding moiety or the pharmacological active agent, the groups providing covalent attachment points for a reactive group of a polymeric reagent include amino (in certain embodiments, a primary amino group), carboxylic acid, and thiol.

Conventional coupling reactions using known reaction conditions can be employed in order to conjugate a polymeric reagent to the binding moiety and the pharmacological agent. Such reactions and conditions are explained in the relevant literature as well as in many of the articles, patents, and patent publications cited herein.

Typical of one approach is a reductive amination reaction used, for example, to conjugate primary amines with a polymer functionalized with a ketone or aldehyde. In this approach, the primary amine of a binding moiety or or pharmacologically active agent reacts with the carbonyl group of the aldehyde or ketone, thereby forming a Schiff base. The Schiff base, in turn, can then be reductively converted in the presence of sodium borohydride to form a stable conjugate. At lower pHs, selective reactions at the N-terminus are possible.

Other polymeric reagents are available that react with groups of a binding moiety and or pharmacologically active agent. For example, if the polymeric reagent is end-functionalized or “activated” with a hydroxyl group, an ester linkage can be formed by reacting the hydroxy-terminated polymeric reagent with a carboxylic acid group-containing binding moiety or pharmacologically active agent. Moreover, if the polymeric reagent is functionalized with a thiol group, a thioester linkage will form between the thiol of the polymeric reagent and the carboxylic acid group-containing pharmacologically active agent.

Additional polymeric reagents include those containing a hydrazide moiety. Such hydrazide-containing polymeric reagents are also useful for conjugation to any available carboxyl group of a binding moiety or pharmacologically active agent. An example of such a polymeric reagent includes a polymeric reagent having the following structure:

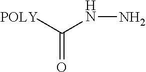

where POLY is a polymer.

Polymeric reagents are known that react with thiol groups (such as in a cysteine residue) of binding moiety and pharmacologically active agent selected from the group consisting of small molecule drugs. Nonlimiting examples of specific polymeric reagents useful for forming covalent linkages with available thiol groups include an N-maleimidyl polymeric reagent as described in U.S. Pat. No. 5,739,208 and in International Patent Publication No. WO 01/62827.

Typically, the weight-average molecular weight of the water-soluble non-peptidic polymer in the conjugate is from about 100 Daltons to about 150,000 Daltons. Exemplary ranges, however, include weight-average molecular weights in the range of greater than 5,000 Daltons to about 100,000 Daltons, in the range of from about 6,000 Daltons to about 90,000 Daltons, in the range of from about 10,000 Daltons to about 85,000 Daltons, in the range of greater than 10,000 Daltons to about 85,000 Daltons, in the range of from about 20,000 Daltons to about 85,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons. In certain embodiments, the weight-average molecular weight of the water-soluble non-peptidic polymer in the conjugate is from about 2,000 to about 25,000 and in certain embodiments, from about 4,000 to about 20,000.

Exemplary weight-average molecular weights for the water-soluble non-peptidic polymer include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. Branched versions of the polymer (e.g., a branched 40,000 Dalton water-soluble polymer comprised of two 20,000 Dalton polymers) having a total molecular weight of any of the foregoing can also be used.

When used as the polymer, PEGs will typically comprise a number of (OCH₂CH₂) monomers [or (CH₂CH₂O) monomers, depending on how the PEG is defined]. As used throughout the description, the number of repeating units is identified by the subscript “n” in “(OCH₂CH₂)ₙ.” Thus, the value of (n) typically falls within one or more of the following ranges: from 2 to about 4000, from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

Although many examples of polymeric reagents have been described, additional polymeric reagents are available for forming the compounds described herein. Thus, the invention is not limited to the compounds formed from the presently described polymeric regents. Furthermore, the invention is not limited with respect to any specific weight average molecular weight as a wide range of molecular weights are possible.

In certain embodiments, a spacer moiety (e.g., “X¹” and “X²”) comprises an ether, amide, urethane, amine, thioether, urea, or a carbon-carbon bond. The spacer moiety may less also comprise (or be adjacent to or flanked by) other atoms, as described further below.

In certain embodiments, a spacer moiety may be any of the following: “—” (i.e., a covalent bond, that may be stable or degradable), —O—, —NH—, —S—, —C(O)—, —C(O)O—, —OC(O)—, —CH₂—C(O)O—, —CH₂—OC(O)—, —C(O)O—CH₂—, —OC(O)—CH₂—, C(O)—NH, NH—C(O)—NH, O—C(O)—NH, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂, —CH₂—CH₂—NH—C(O)—CH₂—CH₂, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, bivalent cycloalkyl group, —N(R⁶)—, R⁶ is H or an organic radical selected from the group consisting of alkyl, substituted alkyl, alkenyl, substituted alkenyl, alkynyl, substituted alkynyl, aryl and substituted aryl. Additional spacer moieties include, acylamino, acyl, aryloxy, alkylene bridge containing between 1 and 5 inclusive carbon atoms, alkylamino, dialkylamino having about 2 to 4 inclusive carbon atoms, piperidino, pyrrolidino, N-(lower alkyl)-2-piperidyl, morpholino, 1-piperizinyl, 4-(lower alkyl)-1-piperizinyl, 4-(hydroxyl-lower alkyl)-1-piperizinyl, 4-(methoxy-lower alkyl)-1-piperizinyl, guanidine, ester, carbonate, and phosphate. In some instances, a portion or a functional group of the drug compound may be modified or removed altogether to facilitate attachment of the oligomer.

For purposes of the present invention, however, a group of atoms is not considered a spacer moiety when it is immediately adjacent to the water-soluble, non-peptidic polymer, and the group of atoms is the same as a monomer of the water-soluble, non-peptidic oligomer such that the group proposed to be a spacer moiety would represent a mere addition of another monomer.

For any given spacer moiety included within the compounds described herein, the spacer moiety can include a releasable bond (such as a hydrolytically releasable bond, e.g., an ester bond). In addition, for any given spacer moiety within the compounds described herein, all bonds within spacer moiety can be non-releasable bonds (e.g., all hydrolytically stable bonds). In certain embodiments the spacer may trigger release based on a reduction or oxidation of the spacer. Various combinations of spacers may be used to achieve a desired effect. As a non-limiting example, for the compounds of the present invention, the spacer moiety may be tuned so that the pharmacologically active agent or binding moiety is released extracellurally (e.g. outside of the cell expressing the particular target of the binding moiety). Alternatively, in instances where the binding results in internalization of the compounds of the invention (or portions thereof) the spacers may be chosen such that the pharmacologically active agent or binding moiety remains bound extracellularly, but may be released intracellularly.

In order to attach the desired binding moiety or pharmacologically active agent to the water-soluble, non-peptidic polymer, a suitable polymeric reagent is allowed to contact the desired species (either the binding moiety or the pharmacologically active agent). Those of ordinary skill in the art know or can determine through routine experimentation whether any given polymeric reagent is suited for forming a conjugate with a specific active agent. For example, species having one or more amine groups will react with polymeric reagents bearing an electrophilic group, as discussed above. In addition, one may simply contact a proposed polymeric reagent with the desired species and determine whether conjugation has been effected by running the reaction mixture through a size-exclusion column; the relatively large conjugates—comprised of both polymer and active agent—will be elute before both the nonconjugated polymeric reagent and active agent.

Often, a heterobifunctional polymeric reagent, e.g., a polymeric reagent bearing a first reactive group and a second reactive group wherein the first reactive group and second reactive group are different. By using a heterobifunctional polymeric reagent, a first reactive group within the heterobifunctional polymeric reagent can be used to react with either the binding moiety or pharmacologically active agent to form an initial conjugate, followed by reaction of the second reactive group (originating from the heterobifunctional reagent and remaining in the initial conjugate) to the other moiety—either the binding moiety or pharmacologically active agent, whichever is absent from the initial conjugate. In this regard, the adaption of orthogonal synthetic strategies [see Baranay et al. (1977) J. Am. Chem. Soc. 116:7363-7365] is one approach for preparing the compounds of the invention using heterobifuntional polymeric reagents. Briefly, this approach relies on a strategy wherein (i) a first reactive group (“rGr¹”) of a polymeric reagent can be activated under conditions A but stable under conditions B, and (ii) a second reactive group (“rGr²”) can be activated under conditions B but stable under conditions A. Individual synthons [such as the binding moiety (“Ab”) and pharmacologically active agent (“Dr”)] carry unique (or substantially disparate amounts of different) functional groups that are specific or selective for one of the first reactive group (“rGr¹”) or second reactive group (“rGr²”). Schematically, the synthetic reaction appears as follows:

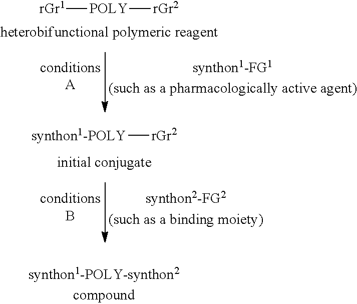

Heterobifunctional polymeric reagents are available commercially and/or are described in the literature.

In one approach, by relying on difference reactive groups of a heterobifunctional polymer (e.g., a polymeric reagent bearing both a sulfhydryl-selective reactive group, such as a maleimide, and an amine-selective reactive group, such as a succinimidyl) it is possible to take advantage of an sulfhydryl-containing binding moiety and an amine-containing, sulfhydryl-lacking pharmacologically active agent. This same principle can be adapted for different pairs of functional groups in the binding moiety/pharmacologically active agent.

In another approach, the compounds of the invention can be prepared by relying on blocking chemistry, wherein attachment of one species (either the binding moiety or the pharmacologically active agent) is attached with a polymeric reagent bearing a blocking or protecting group, followed by removal of the blocking or protecting group using conventional techniques, and subsequent attachment of the other species (the other of the binding moiety or pharmacologically active agent).

Having selected a polymeric reagent and synthon (e.g., a binding moiety or a pharmacologically active agent), the polymeric reagent is typically added to the synthon in an equimolar amount (with respect to the desired number of groups suitable for reaction with the reactive group) or at molar excess. For example, the polymeric reagent can be added to the synthon at a molar ratio of about 1:1 (polymer:synthon), 1.5:1, 2:1, 3:1, 4:1, 5:1, 6:1, 8:1, 10:1, 20:1 or 40:1.

The conjugation reaction is allowed to proceed until substantially no further conjugation occurs, which can generally be determined by monitoring the progress of the reaction over time. Progress of the reaction can be monitored by withdrawing aliquots from the reaction mixture at various time points and analyzing the reaction mixture by SDS-PAGE or MALDI-TOF mass spectrometry, high-performance liquid chromatography (HPLC), or any other suitable analytical method. Once a plateau is reached with respect to the amount of conjugate formed or the amount of unconjugated polymeric reagent remaining, the reaction is assumed to be complete.

Typically, the conjugation reaction takes anywhere from minutes to several hours (e.g., from 5 minutes to 24 hours or more). The resulting population of conjugates is preferably, but not necessarily, purified so as to separate out excess reagents, unconjugated synthon, undesired multi-conjugated species, and free or unreacted polymeric reagent. The resulting population can also be further characterized using analytical methods such as MALDI, capillary electrophoresis, gel electrophoresis, and/or chromatography.

With respect to the resulting conjugates, the conjugates can optionally be purified to obtain/isolate different conjugated species. Once the first of the binding moiety or pharmacologically active agent has been attached to the water-soluble, non-peptidic polymer, the other of the binding moiety or pharmacologically active agent can be attached following a similar technique.

The pharmacologically active agent for coupling to a water-soluble, non-peptidic polymer may possess a free hydroxyl, carboxyl, thio, amino group, or the like (i.e., “handle”) suitable for covalent attachment to the polymer. In addition, the pharmacologically active agent may be modified by introduction of a reactive group, preferably by conversion of one of its existing functional groups to a functional group suitable for formation of a stable covalent linkage between the polymer and the drug.

Certain Affibody® molecules contain a unique C-terminal cysteine that serves as a site for targeted modification. With respect to PEGylation, Affibody® molecules may be modified with sulfhydryl-reactive PEG reagents disclosed herein, including for example maleimide-PEG reagents. Due the presence of the terminal cysteine the Affidbody® molecules may form sulfide-linked covalent dimers. As such, the dimers may need to be reduced prior to PEGylation. Further, it may be necessary to remove the reducing agent prior to PEGylation. Additionally, steps may need to be taken to reduce the tendency of the dimers to reform. In light of above, reduction and PEGylation conditions that reduce the likelihood of Affibody® dimerization while allowing the attachment of PEG to the free thiol group have been developed and are described below.

In order to expose the reactive cysteine, it may be necessary to reduce the dimers. In certain embodiments, reactions of the free Affibody® molecules take place in an environment that prevents formation of disulfide bridges. As such, in certain embodiments, PEGylation of Affibodys takes place by reducing the disulfide linked Affibody® dimers and incubation of the free Affibody® molecules with a thiol reactive PEG reagent. In some embodiments the reduction takes place in the presence of a suitable reducing agent. In certain embodiments, the reducing agent is selected from 2-mercaptoethanol, dithiothreitol (DTT), and tris(2-carboxyethyl)phosphine (TCEP). In certain embodiments, the reducing agent (e.g. DTT) is removed from the free Affibody® molecules prior to PEGylation. In certain embodiments, the reducing agent is removed via ion exchange/reverse phase chromatography. In certain embodiments, the PEGylation step takes place at a pH below about 7.4. In certain embodiments the PEGylation step takes place at a pH between about 3.0 and about 7.0. In certain embodiments, the PEGylation step takes place at a pH between about 5.0 and about 7.0. In certain embodiments, the PEGylation step takes place at a pH between about 5.5 and about 6.5. In certain embodiments, the PEGylation step takes place at a pH of about 6.0. In certain embodiments, the PEGylation step takes place in the presence of EDTA.

The present invention also includes pharmaceutical preparations comprising the compounds provided herein in combination with a pharmaceutical excipient. Thus, the method for making the compositions herein includes the optional step of adding a pharmaceutically acceptable excipient. Generally, the composition itself will be in a solid form (e.g., a precipitate), which can be combined with a suitable pharmaceutical excipient that can be in either solid or liquid form. For example, the compositions (and corresponding pharmaceutical preparations) can be provided in a lyophilized form or in a solution.

Depending on the intended mode of administration, the composition may be a liquid, semi-solid or solid. Exemplary liquids include a suspension, a solution, an emulsion, and a syrup, which can be formulated for administration to a patient. Exemplary semi-solids include gels which can be administered “as is” or formulated (e.g., into a gel-cap) for administration to a patient. Exemplary solids include granules, pellets, beads, powders, which can be administered “as is” or formulated into one or more of the following for administration to a patient: a tablet; a capsule; a caplet; a suppository; and a troche. In certain embodiments, the composition will be in a unit dosage form to thereby provide a unit dosage suitable for single administration of a dosage of each active component in the unit dosage form. Suitable pharmaceutical compositions and dosage forms may be prepared using conventional methods known to those in the field of pharmaceutical formulation and described in the pertinent texts and literature, e.g., in Remington's Pharmaceutical Sciences: 18^{th} Edition, Gennaro, A. R., Ed. (Mack Publishing Company; Easton, Pa.; 1990).

Oral dosage forms include tablets, capsules, caplets, gel caps, troches, solutions, suspensions, and syrups. Tablets and capsules represent the most convenient oral dosage forms.

Tablets can be manufactured using standard tablet processing procedures and equipment. Preferred techniques for forming tablets include direct compression and granulation. In addition to the active agents, tablets will generally contain inactive, pharmaceutically acceptable carrier materials such as binders, lubricants, disintegrants, fillers, stabilizers, surfactants, coloring agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

In some instances, the tablet can be in the form of a uniform tablet. In uniform tablets, the formulation used in preparing the tablet is a substantially homogenous mixture of active agents and one or more pharmaceutical excipient (e.g., diluent). The formulation is then used to make tablets using a suitable tableting process to thereby result in a tablet that is substantially homogenous throughout the tablet.

In still other instances, the tablet can also take the form of a layered tablet (of one, two, three or more layers). The method for manufacturing the layered tablet can include combining two different formulations (e.g., one formulation containing the opioid agonist and another containing the polymer-opioid conjugate) and compressing the two together to form the tablet. Multiple layered tablets of three or more layers are also possible and can be formed, for example, in a similar manner by combining three or more distinct formulations and followed by compression.

Optionally, a barrier layer can be included in the layered tablet. One approach for incorporating a barrier layers involves forming a compressed first layer of a first formulation (e.g., a formulation containing a first active agent) wherein the compress layers has one exposed surface, coating the exposed surface with a material (e.g., a material that is substantially impermeable to thereby prevent physical interaction between adjacent layers) to form a coated surface, and contacting the coated surface with a second formulation (e.g., a second formulation containing a second active agent), and compressing the second formulation and coated surface to form a layered tablet having a barrier layer included therein.

Capsules are also oral dosage forms, in which case the composition may be encapsulated in the form of a liquid, semi-solid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington's Pharmaceutical Sciences, supra, which describes materials and methods for preparing encapsulated pharmaceuticals.

The pharmaceutical preparations can also be administered via injection and can therefore be liquid solutions or suspensions immediately prior to administration. Other modes of administration are also included, such as pulmonary, rectal, transdermal, transmucosal, oral, intrathecal, subcutaneous, intra-arterial, and so forth. Advantageously, the pharmaceutical preparation (as well as the composition) can be provided in a unit dosage form. In addition, the pharmaceutical preparation (as well as the composition) can be used in a vial (e.g., a glass vial or a plastic vial) or in a syringe to provide a prefilled syringe.

As previously described, the pharmaceutical preparation can be injected parenterally by intravenous injection, or by intramuscular or by subcutaneous injection. Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others. Typically, the preparation is provided in a dry form (e.g., lyophilized form) for storage and is reconstituted with a suitable liquid diluent. Preferred liquid diluents are selected from the group consisting of bacteriostatic water for injection, dextrose 5% in water, phosphate-buffered saline, Ringer's solution, saline solution, sterile water, deionized water, and combinations thereof.

Exemplary excipients of the pharmaceutical preparations include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, sorbitol (glucitol), pyranosyl sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The preparation may also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the preparation as well. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfate, sodium formaldehyde sulfoxylate, sodium metabisulfite, and combinations thereof.

A surfactant may be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations.

Acids or bases may be present as an excipient in the preparation. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The pharmaceutical preparations encompass all types of formulations. The amount of the active agents (i.e., compounds of the present invention) in the composition will vary depending on a number of factors, but will optimally be a therapeutically effective dose of each active agent when the composition is stored in a unit dose form. A therapeutically effective dose for each active agent can be determined experimentally by repeated administration of increasing amounts of the active agent in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 2%-98% by weight, more preferably from about 5-95% by weight of the excipient, with concentrations less than 30% by weight most preferred.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A.H., Handbook of Pharmaceutical Excipients, 3^{rd} Edition, American Pharmaceutical Association, Washington, D.C., 2000.

The invention also provides a method for administering the pharmaceutical preparation as provided herein to a patient suffering from a condition that is responsive to treatment with the compound. The method comprises administering a therapeutically effective amount of the compound (e.g. provided as part of a pharmaceutical composition). The method of administering may be used to treat any condition that can be remedied or prevented by administration of the particular compound. Those of ordinary skill in the art appreciate which conditions a specific compound can effectively treat (and typically is a same condition for which agent selected from the group consisting of small molecule drugs, toxins and radioactive substances is used to treat). For example, when the binding agent is a HER2 binding Affibody® and the pharmacologically active agent is chosen from one that may treat the underlying condition associated with HER2 overexpression, the compound of the present invention may be used to treat that underlying disease associated with HER2 overexpression. By means of a non-limiting example, conditions associated with HER2 over expression include breast cancer, ovarian cancer, pancreatic cancer, stomach cancer, salivary cancer, lung cancer, and esophageal cancer. In such cases, by means of a non-limiting example, the pharmacologically active agent is chosen from docetaxel, paclitaxel, anthracylcin, topotecan, gemcitabine and lapatinib.

The actual dose to be administered will vary depend upon the age, weight, and general condition of the subject as well as the severity of the condition being treated, the judgment of the health care professional, and compound being administered. Therapeutically effective amounts are known to those skilled in the art and/or are described in the pertinent reference texts and literature. Generally, a therapeutically effective amount will range from about 0.001 mg to 1.0 g, in certain embodiments in doses from 0.01 mg/day to 750 mg/day, in certain embodiments from about 0.10 mg/day to 500 mg/day. These values may include the amount of the compound of the present invention. These values may also include the effective amount of the pharmacologically active agent in the compound of the present invention.

The unit dosage of any given compound (in certain embodiments, provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing of the composition is halted.

The compounds for use in connection with the invention described herein will typically possess a measurable degree of bioactivity. For instance, such compounds are typically characterized as having a bioactivity satisfying one or more of the following percentages relative to that of the unconjugated pharmacologically active agent: at least about 2%, at least about 5%, at least about 10%, at least about 15%, at least about 25%, at least about 30%, at least about 40%, at least about 50%, at least about 60%, at least about 80%, at least about 85%, at least about 90%, at least about 95%, at least about 97%, at least about 100%, and more than 105% (when measured in a suitable model, such as those well known in the art).

The bioactivity of the compounds disclosed herein can be determined by methods known to one of skill in the art. For example, the compounds may be analyzed using methods that are known to measure the bioactivity of the unconjugated pharmacologically active agent. As a non-limiting example, the bioactivity of compounds of the present invention where the pharmacologically active agent is an anti-cancer agent may be analyzed in an appropriate tumor xenograft model. Further, the model used may also depend on the particular binding agent used. For example, in embodiments where the binding agent is a HER2 Affibody® and the pharmacologically active agent is an anti-cancer agent, the compounds may be analyzed in a tumor xenograft model, where the tumor cells are of the type that overexpresses the HER2 receptor. Regardless of the binding moiety, in certain embodiments the compounds may be tested in a model where the target of the binding moiety is expressed.

The ability of the compounds of the present invention to bind to the target of the binding moiety may be assessed by methods known to one of skill in the art. For example, the compound may include a labeling agent either in addition to or in place of the pharmacologically active agent and the localization of the compounds may be analyzed in vivo. Additionally, in vitro binding may be analyzed by methods known in the art. Additional methods are described in the Examples below. In view of the above, compounds of the present invention wherein the pharmacologically active agent is replaced with a labeling agent are also within the scope of the disclosure.

It is to be understood that while the invention has been described in conjunction with the specific embodiments thereof, that the foregoing description as well as the experimental that follow are intended to illustrate and not limit the scope of the invention. Other aspects, advantages and modifications within the scope of the invention will be apparent to those skilled in the art to which the invention pertains. All articles, books, patents, patent publications and other publications referenced herein are hereby incorporated by reference in their entireties.

### EXPERIMENTAL

The practice of the invention will employ, unless otherwise indicated, conventional techniques of organic synthesis and the like, which are understood by one of ordinary skill in the art and are explained in the literature. In the following examples, efforts have been made to ensure accuracy with respect to numbers used (e.g., amounts, temperatures, and so forth), but some experimental error and deviation should be accounted for.

### Example 1

### Preparation of PEGylated Affibody® Molecules

PEGylated Affibody® molecules were prepared as according to the following procedure.

### A. Reduction of Affibody® Molecules

Affibodys were purchased from Affibody AB. Two different HER2 Affibodys were purchased, which are designated Affibody A and Affibody B. Affibody stock solution (800 μL of a 5 mg/mL solution of Affibody A or Affibody B) in water, 80 μL of 1 M DTT (Dithiothreitol), 200 μL of 1 M HEPES (pH 7.4) and 2.92 mL water were mixed in a 15 mL polypropylene conical tube. The reaction mixture was incubated at 37° C. for 1 hour. The reactions were diluted 1:5 with 10 mM NaOAc, pH 4.0, and the excess of DTT was removed by cation exchange chromatography (5 mL HiTrap SP Sepharose HP column, GE Healthcare). Buffer A was 10 mM NaOAc, pH 4.0, and Buffer B was 10 mM NaOAc, 1 M NaCl, pH 4.0. After loading, the column was washed in buffer A for 3 column volumes and the reduced affibodys were eluted using a linear gradient of 0 to 100% B in 10 column volumes at a flow rate of 5 mL/min.

Fractions containing Affibodys were pooled and concentrated over a reversed phase CG71S column (5 mm×100 mm) equilibrated with 0.5% acetic acid. After loading, the column was washed with 0.5% acetic acid until both the UV absorbance and conductivity baseline stabilized. The Affibody® proteins were eluted with 60% of acetonitrile containing 0.5% acetic acid. The fractions containing pure affibodys were collected, lyophilized and stored at −80° C.

### B. PEGylation of Affibody® Molecules

Stock solutions of 2 mg/mL (286 μM) reduced Affibody® B and 2 mg/mL (303 μM) reduced Affibody® A were prepared in 20 mM MES/1 mM EDTA (pH 6.0). A 2.5 mM stock solution of each PEG reagent (20 kD mPEG-m-MAL, 40 kD mPEG-ru-MAL, 5 kD mPEG-MAL, 20 kD mPEG-MAL, 30 kD mPEG-MAL and Cy5.5-20 kD PEG-MAL) was made in the same buffer used to dissolve the Affibodys. Structures of the PEG reagents are depicted below. The values adjacent to the repeating ethylene oxide units, when listed as a kD value, refer to the average molecular weight of the repeating unit, as opposed to the number of ethylene oxide units.

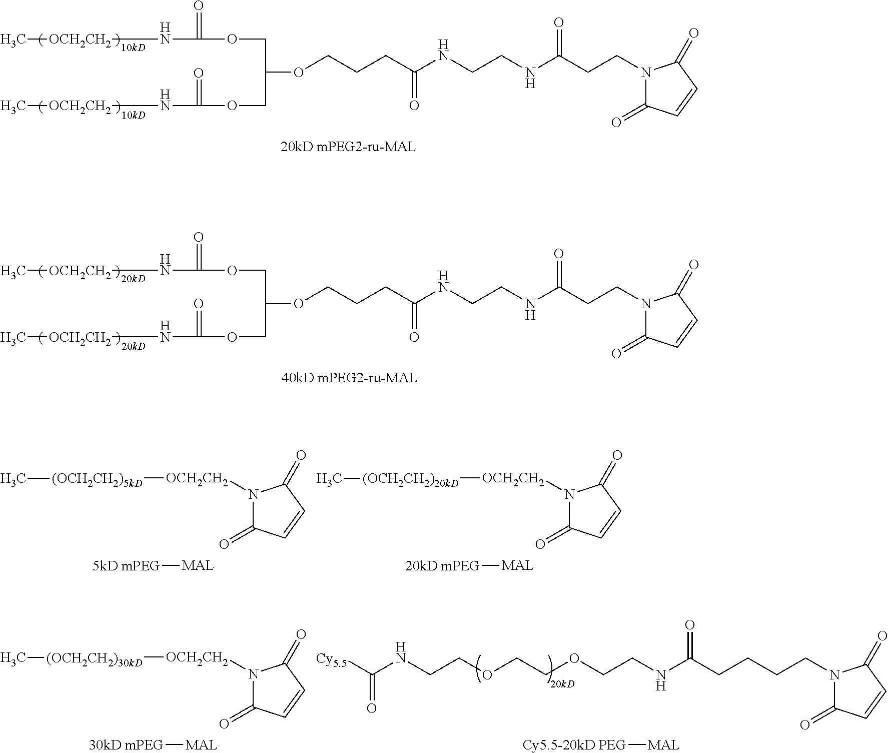

Typical PEGylation reaction was carried out as follows:

Affibody® stock solution (300 μl) was transferred to an Eppendorf tube and 232 μL or 210 μl, of 20 mM MES/1 mM EDTA (pH 6.0) buffer was added to the solution of Affibody® B or Affibody® A, respectively. PEGylation was initiated by drop-wise addition of 68 μL of PEG stock solution to Affibody® B or 90 μL of PEG stock solution to Affibody® A. The reaction mixtures were incubated at room temperature for 3 hours. The resultant reaction mixture contained 1 mg/mL peptide and 2.0 mol equivalents of PEG (with respect to peptide). The reaction yields were determined by reversed phase HPLC to be over 95% for Affibody® B and 85-90% for Affibody® A.

The PEGylated Affibody® conjugates were purified from the reaction mixture by cation exchange chromatography using a 5 mL Hi Trap SP Sepharose HP column (GE Healthcare). The buffers used for purification were: Buffer A: 10 NaOAc, pH 4.0 and Buffer B: Buffer A+1 M NaCl. The PEGylation reaction mixtures were diluted with 4 volumes of buffer A and load onto the column equilibrated in buffer A. Unbound substances were washed off the column with 5 column volumes of buffer A. The PEGylated and non-PEGylated affibodys were eluted from the column using a linear gradient of 0 to 100% B over 10 column volumes at a flow rate of 5 mL/min.

Fractions containing PEGylated affibodys were pooled and concentrated over a reversed phase CG71S column (5 mm×100 mm) equilibrated with 0.5% acetic acid. After loading, the column was washed with 0.5% acetic acid until both the UV absorbance and conductivity baseline stabilized. The conjugates were eluted with 60% of acetonitrile containing 0.5% acetic acid. The fractions containing pure PEGylated Affibodys were collected and lyophilized. The lyophilized conjugates were reconstituted in water and stored frozen at −80° C.

The purity of PEG-Affibody® conjugates was >95% by RP-HPLC analysis. The mobile phases were: A, 0.09% TFA in water and B, 0.04% TFA in acetonitrile. An Intrada WP-RP C18 column (3×150 mm) was used with a flow rate of 0.5 ml/min and a column temperature of 45° C. Detection was carried out at 280 nm. The column was equilibrated with Buffer A and conjugate separation was achieved using the gradient timetable shown below:

| | Time | % A | % B |
|---|---|---|---|
| 1 | 0.00 | 90.0 | 10.0 |
| 2 | 5.00 | 90.0 | 10.0 |
| 3 | 55.00 | 40.0 | 60.0 |
| 4 | 60.00 | 40.0 | 60.0 |
| 5 | 61.00 | 90.0 | 10.0 |
| 6 | 65.00 | 90.0 | 10.0 |

Additionally, the masses of the compounds prepared above were analyzed by MALDI-TOF and found to be in the expected range. Representative masses for the singly charged compounds were measured to be:

| Compound | Mass |
|---|---|
| [mono]-[20kD mPEG-ru-MAL]-[Affibody ® A] | 28149 Da |
| [mono]-[40kD mPEG-ru-MAL]-[Affibody ® A] | 46239 Da |
| [mono]-[5kD mPEG-MAL]-[Affibody ® A] | 11808 Da |
| [mono]-[20kD mPEG-MAL]-[Affibody ® A] | 27608 Da |
| [mono]-[30kD mPEG-MAL]-[Affibody ® A] | 38193 Da |
| [mono]-[Cy5.5-20kD PEG-MAL]-[Affibody ® A] | 28301 Da |
| [mono]-[20kD mPEG-ru-MAL]-[Affibody ® B] | 28966 Da |
| [mono]-[40kD mPEG-ru-MAL]-[Affibody ® B] | 47708 Da |
| [mono]-[5kD mPEG-MAL]-[Affibody ® B] | 12202 Da |
| [mono]-[20kD mPEG-MAL]-[Affibody ® B] | 28265 Da |
| [mono]-[30kD mPEG-MAL]-[Affibody ® B] | 39388 Da |
| [mono]-[Cy5.5-20kD PEG-MAL]-[Affibody ® B] | 29010 Da |

### Example 2

### Determination of Affinity for HER-2 Receptor of Affibody® Conjugates

Equipment: ProteOn XPR36 (BioRad)

Materials: GLM sensor chips were from BioRad. Anti-human mAb from Invitrogen (Cat. Number H10500). RhErbB2/Fc was from R&D Systems (Cat. Number 1129-ER)

A new GLM sensor chip was docked and the system primed with hepes buffered saline (HBS). Anti-human mAb was amine coupled using sulfo-NHS/EDC activation for 5 minutes followed by an injection of anti-human mAb at 10 ug/ml in 10 mM NaOAc (pH 5.0). Surfaces were then blocked with 1 M ethanolamine pH 8.5. This resulted in the coupling of ˜8,500 RU of mAb onto the surface.

50 ug of rhErbB2/Fc was dissolved into 250 of HBS-p with 0.1 mg/ml BSA. The receptor was then diluted to 2, 0.6, 0.2, 0.07, and 0.024 ug/ml and captured for 5 minutes over the anti-Fc surface. Capture levels ranged from 900, 500, 300, 100 to 50 RU.

Samples were each tested at 100 nM as the highest concentration in a 3-fold dilution series. The response data from each different density receptor surface were globally fitted to determine the rate constants summarized in Table 1.

TABLE 1
Binding Constants Determined at 25 degrees C., pH 7.5.

| | ka (M−1s−1) | kd (s−1) | KD (pM) |
|---|---|---|---|
| [mono]-[20kD mPEG-ru-MAL]-[Affibody ® B] | 3.2(3)e6 | 0.0010(5) | 300(200) |
| [mono]-[30kD mPEG-MAL]-[Affibody ® B] | 2.37(1)e7 | 0.001021(2) | 43.2(2) |
| [mono]-[20kD mPEG-ru-MAL]-[Affibody ® A] | 5.22(3)e6 | 0.00132(4) | 254(9) |
| [mono]-[40kD mPEG-ru-MAL]-[Affibody ® A] | 8.04(5)e6 | 0.001408(4) | 175(1) |
| [mono]-[5kD mPEG-MAL]-[Affibody ® A] | 2.12(3)e7 | 9.08(5)e−4 | 42.8(5) |
| [mono]-[20kD mPEG-MAL]-[Affibody ® A] | 1.84(2)e7 | 0.001159(4) | 63.0(5) |
| [mono]-[30kD mPEG-MAL]-[Affibody ® A] | 3.58(4)e7 | 0.001142(5) | 31.9(3) |
| [mono]-[Cy5.5-20kD PEG-MAL]-[Affibody ® B] | 1.03(1)e7 | 0.001066(4) | 103.4(9) |

(The number in parentheses represents the standard error in the last reported digit)

A new GLM sensor chip was docked and the system primed with HBS. Anti-human mAb was amine coupled using sulfo-NHS/EDC activation for 5 minutes followed by an injection of anti-human mAb at 10 ug/ml in 10 mM NaOAc (pH 5.0). Surfaces were then blocked with 1 M ethanolamine pH 8.5. This resulted in the coupling of ˜8,000 RU of mAb onto the surface.

50 ug of rhErbB2/Fc was dissolved into 250 of HBS-p with 0.1 mg/ml BSA. The receptor was then diluted to 6, 3, 1, 0.33, and 0.11 ug/ml and captured for 5 minutes over the anti-Fc surface. Capture levels ranged from 1200, 900, 400, 150 to 50 RU.

Samples were each tested at 100 nM as the highest concentration in a 3-fold dilution series. The response data from each different density receptor surface were globally fitted to determine the rate constants summarized in Table 2.

TABLE 2
Binding Constants Determined at 25 Degrees C., pH 7.5.

| | ka (M−1s−1) | kd (s−1) | KD (pM) |
|---|---|---|---|
| Affibody ® B | 2.053(9)e7 | 0.00190(1) | 92.6(6) |
| Affibody ® A | 2.28(5)e7 | 0.0048(1) | 209.2(7) |
| [mono]-[40kD mPEG-ru-MAL]- | 3.34(1)e6 | 0.001130(2) | 338.8(9) |
| [Affibody ® B] | | | |
| [mono]-[5kD mPEG-MAL]- | 2.47(1)e7 | 0.001009(3) | 40.9(1) |
| [Affibody ® B] | | | |
| [mono]-[20kD mPEG-MAL]- | 7.99(2)e6 | 0.001029(1) | 128.9(2) |
| [Affibody ® B] | | | |
| [mono]-[Cy5.5-20kD PEG- | 4.81(1)e6 | 9.91(2)e−4 | 205.9(5) |
| MAL]-[Affibody ® A] | | | |

(The number in parentheses represents the standard error in the last reported digit)

### Example 3

### Imaging Study on Affibody® Biodistribution in Mouse Tumor Xenograft Model

To assess localization of anti-HER2 Affibody® conjugates to tumors, SKOV-3 tumor cells, which express high levels of HER2 protein, were injected subcutaneously into the flank regions of athymic, nude mice at 5×10⁶ cells/100 ul for each flank. Female athymic nude mice (Hsd:Athymic Nude-Foxn 1nu) were obtained from Harlan Laboratory and housed in testing facility for at least 3 days for acclimation. KB and SKOV-3 tumor cell lines were purchased from ATCC and cultured in 37 C incubator. Tumor cell suspension was prepared in serum-free medium at 1×10⁷/mL for KB cell and 5×10⁷/mL for SKOV-3 cells. 1×10⁶ of KB cells or 5×10⁶ SKOV-3 cells were injected subcutaneously into nude mice in the lower thoracic flanking region. The tumors were allowed to grow for 2 weeks (KB) or 6 (SKOV-3) weeks before biodistribution study.

IRDye-800 labeled Affibody® compounds, Cy5.5-labeled 20 kD PEG, or Cy5.5-20 kD PEG-Affibody® conjugates were diluted with 0.9% NaCl saline solution to 0.3 nmol/100 ul. Testing compounds were dosed with a single IV through tail vein at 0.3 nmol/animal.

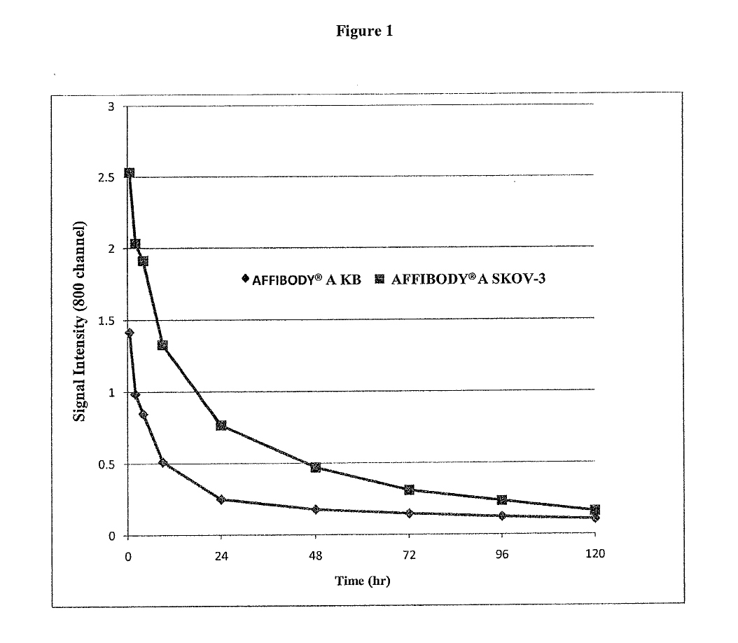

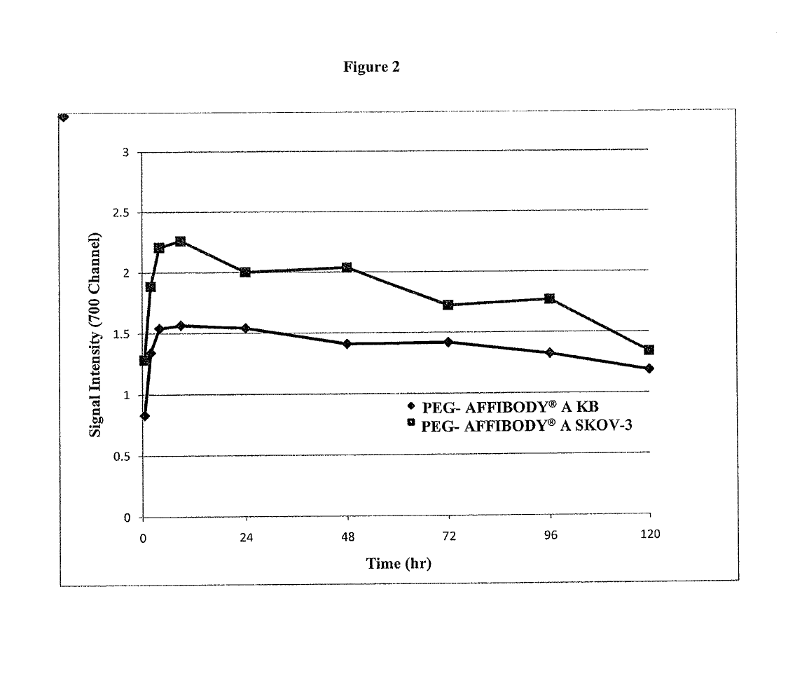

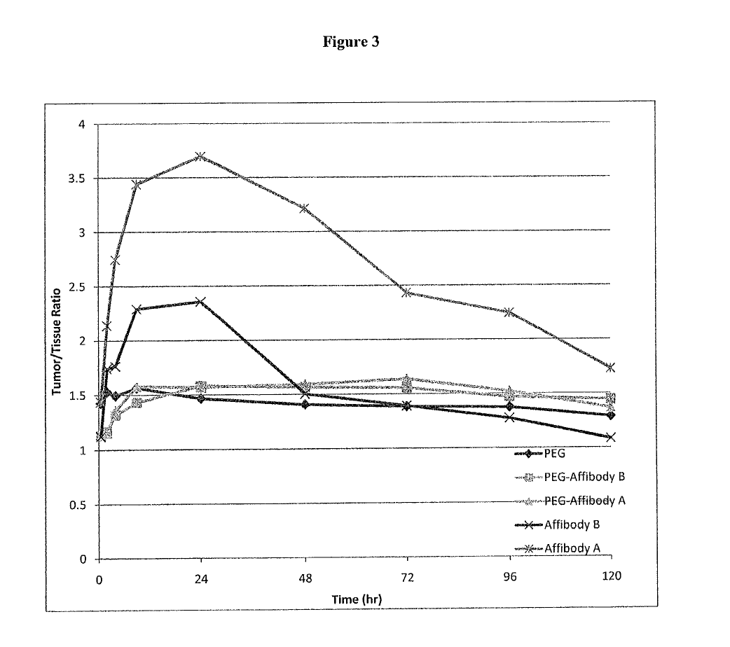

At different times after dosing, the nude mice were anesthetized with isoflurane and their whole body images were recorded using Pearl near-infrared imager (LI-COR). Signal intensity in the tumor, normal tissue region, and kidney were analyzed and plotted. Additionally the ratio signal intensity in the rumor region over non-tumor region was determined. The results are represented in FIGS. 1-2. FIG. 1 is a plot of the signal intensity of IRDye-800 labeled Affibody A in tumor cells SKOV-3 and KB vs. time. FIG. 2 is a plot of the signal intensity of Cy5.5-20 kD PEG-Affibody A in tumor cells SKOV-3 and KB vs. time. FIG. 3 is a plot of the ratio of signal intensity for SKOV-3 tumors over normal cells vs. time for the tested articles (IRDye-800 labeled Affibody® compounds (PEG-Affibody A, PEG Affibody B), Cy5.5-labeled 20 kD PEG (PEG), or Cy5.5-20 kD PEG-Affibody® (PEG Affibody A, PEG Affibody B).

Based on these studies, the Affibody® molecules (A and B) showed specific SKOV-3 tumor accumulation. PEGylation prolonged the Affibody SKOV-3 tumor accumulation, but not in the KB tumor, suggesting the PEGylated Affibody® accumulation in SKOV-3 is HER2 binding specific.

### Example 4

### Prophetic Preparation of Ab-X¹-POLY-X²-Dr

A compound of the general formula “Ab-X¹—POLY-X²-Dr” having physiologically cleavable (i.e., releaseable) linkages is prepared. This example has only stable linkages.

The Ab-X¹-POLY-X²-Dr construct is produced in a conjugation reaction with a binding moiety (i.e., Affibody) (“Ab”) and a homo- or hetero-bifunctional polymeric reagent containing a reactive group on both ends, rGr¹-Poly-rGr². A hetero-bifunctional polymeric reagent that can be utilized if the binding moiety (“Ab”) contains a thiol group is an alpha-succinimidyl, omega-maleimidyl, polyethyleneglycol, “NHS-PEG-MAL” (See, for example, U.S. Pat. Nos. 6,448,369 and 6,602,498) in which the succinimidyl group (corresponding, in this instance, to “rG¹”) reacts with an amine group on the pharmacologically active agent (“Dr”). The reaction between the polymeric reagent “NHS-PEG-MAL” and Dr is performed with a 5-fold molar excess of NHS-PEG-MAL over Dr at pH 7.4 for six hours at 37° C. The size of the polymeric component should be less than 2,000 Da if close proximity of the binding protein and pharmacologically active agent is desired. The polymeric component should be at least 20,000 Da if a construct larger than the renal filtration size limit is desired. The resulting intermediate corresponds to “rGr²-POLY-X²-Dr”. Additionally, the reagent NHS-X²—PEG-MAL, may be used in a similar manner.

The rGr²-POLY-X²-Dr construct containing the maleimidyl functional group (MAL-POLY-X²-Dr) can then be reacted with the binding moiety (“Ab”) containing the thiol group to produce the final product, Ab¹-X¹-POLY-X²-Dr. The reaction is performed with a 2-fold molar excess of the binding moiety (“Ab”) over rGr²-POLY-X²-Dr at pH 6.0.

### Example 5

### Synthesis of MAL-PEGn-BA-Docetaxel

MAL-PEGn-BA-docetaxel was prepared according to the following procedure.

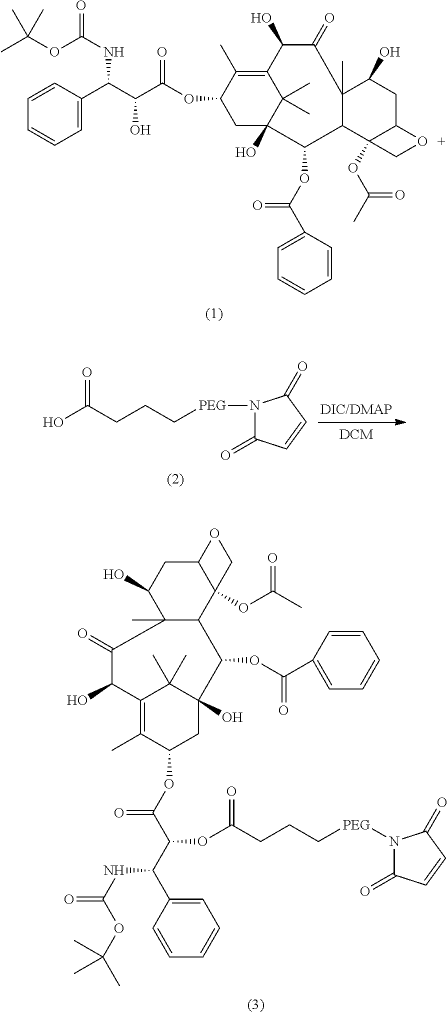

Docetaxel (1) (0.606 g, 0.75 mmol), 4-dimethylaminopyridine (DMAP) (0.009 g, 0.075 mmol), and diisopropylcarbodiimide (DIC) (0.028 g, 0.225 mmol) were dissolved in 10 mL of dichloromethane (DCM). The mixture was cooled with an ice-bath under stirring for 5 min. before solid MAL-PEGn-BA (2) (where PEG is a polyethylene glycol molecule (—OCH2CH2)n having an average MW of 2 kD or 20 kD, 0.075 mmol) was added potionally. The mixture was stirred for 1 hour and then the ice-bath was removed. The reaction was allowed to continue overnight. The reaction solution was concentrated to remove ⅔ of the DCM and then transferred slowly into 100 mL of isopropyl alcohol/ethyl ether (1:1) under stirring. A white solid was collected and washed with ethyl ether. The precipitation was repeated once. 1.44 g product (3) (yield, 92%) was obtained after dried under vacuum.

MAL-2 kD PEG-BA-docetaxel (3) ¹H NMR (500 MHz, CDCl₃): δ 8.11 (d, 2H), 7.62 (m, 1H), 7.50 (m, 2H), 7.40 (m, 2H), 7.26 (m, 2H), 6.70 (s, 2H), 6.28 (m, 1H), 5.70 (d, 1H), 5.50 (m, 2H), 5.35 (s, 1H), 5.25 (s, 1H), 4.97 (d, 1H), 4.34 (d, 1H), 4.30 (m, 1H), 4.20 (m, 2H), 3.91 (d, 1H), 3.90-3.30 (m, ˜210H), 2.60 (m, 1H), 2.43 (s, 3H), 2.38-2.10 (m, 3H), 1.95-1.80 (m, 4H), 1.76 (s, 3H), 1.70 (s, 1H), 1.32 (s, 9H), 1.25 (s, 3H), 1.12 (s, 3H). MALDI-TOF: Mn 3121
MAL-20 kD PEG-BA-docetaxel (3) ¹H NMR (500 MHz, CDCl₃): δ 8.11 (d, 2H), 7.62 (m, 1H), 7.50 (m, 2H), 7.40 (m, 2H), 7.26 (m, 2H), 6.70 (s, 2H), 6.28 (m, 1H), 5.70 (d, 1H), 5.50 (m, 2H), 5.35 (s, 1H), 5.25 (s, 1H), 4.97 (d, 1H), 4.34 (d, 1H), 4.30 (m, 1H), 4.20 (m, 2H), 3.91 (d, 1H), 3.90-3.30 (m, ˜2006H), 2.60 (m, 1H), 2.43 (s, 3H), 2.38-2.10 (m, 3H), 1.95-1.80 (m, 4H), 1.76 (s, 3H), 1.70 (s, 1H), 1.32 (s, 9H), 1.25 (s, 3H), 1.12 (s, 3H). MALDI-TOF: Mn 21633

### Example 6

### Synthesis of Affibody A-MAL-nkD PEG-BA-docetaxel

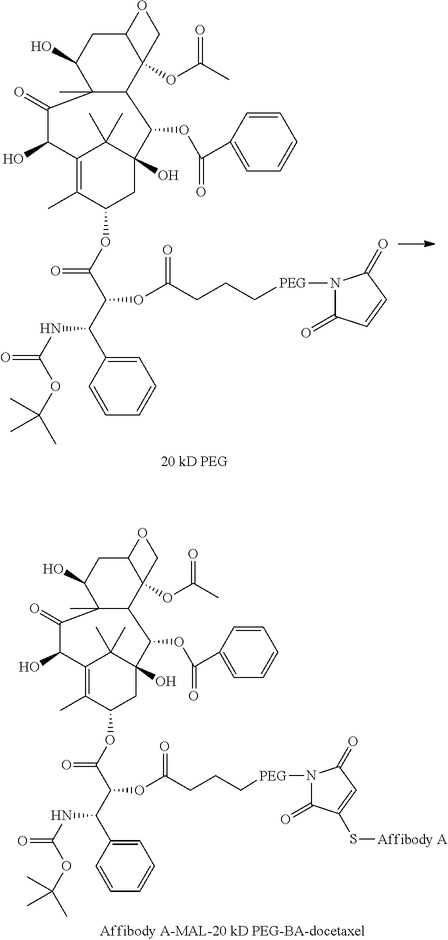

Stock solution of 2 mg/mL (303 μM) reduced Affibody A was prepared in 20 mM MES/1 mM EDTA (pH 6.0). A 1 mM stock solution of MAL-20 Kd PEG-BA-docetaxel was made in the same buffer used to dissolve the Affibody A. The Affibody A stock solution (10.8 mL) was transferred to a 50 mL centrifuge tube containing 2.6 mL of 20 mM MES/1 mM EDTA (pH 6.0) buffer. Drop-wise addition of 8.2 mL of MAL-20 Kd PEG-BA-docetaxel stock solution to the Affibody A solution followed. The reaction mixtures were incubated at room temperature for 2 hours. The resultant reaction mixture contained 1 mg/mL peptide and 2.5 mol equivalents of MAL-20 Kd PEG-BA-docetaxel.

The Affibody A-MAL-20 kD PEG-BA-docetaxel conjugate was purified from the reaction mixture by cation exchange chromatography using SP-HP Sepharose (GE Healthcare) packed into a XK26 column (2.6×15 cm). The buffers used for purification were: Buffer A: 10 mM NaOAc, pH 4.0 and Buffer B: Buffer A+1 M NaCl. The reaction mixture was diluted with 10 volumes of buffer A and loaded onto the column equilibrated in buffer A. Unbound substances were washed off the column with 5 column volumes of buffer A. The product and free Affibody A were eluted from the column using a linear gradient of 0 to 100% B over 10 column volumes at a flow rate of 8 mL/min.

Fractions containing Affibody A-MAL-20 kD PEG-BA-docetaxel were pooled and concentrated over a reversed phase CG71S (Amberchrom) column (1.5×19 cm) equilibrated with 0.5% acetic acid. After loading, the column was washed with 0.5% acetic acid until both the UV absorbance and conductivity baseline stabilized. The Affibody A-MAL-20 kD PEG-BA-docetaxel was eluted with 60% of acetonitrile containing 0.5% acetic acid. The fractions containing Affibody A-MAL-20 kD PEG-BA-docetaxel were collected and lyophilized. The lyophilized conjugates were reconstituted in 10 mM NaOAc (pH 4.0)/150 mM NaCl and stored frozen at −80° C.

The purity of Affibody A-MAL-20 kD PEG-BA-docetaxel conjugate was 99% by RP-HPLC analysis. The mobile phases were: A, 0.09% TFA in water and B, 0.04% TFA in acetonitrile. An Intrada WP-RP C18 column (3×150 mm) was used with a flow rate of 0.5 ml/min and a column temperature of 45° C. Detection was carried out at 215 nm. The column was equilibrated with Buffer A and the separation of the conjugates Affibody A-MAL-20 kD PEG-BA-docetaxel and was achieved using the gradient shown in Table 3.

TABLE 3

| | Time | % A | % B |
|---|---|---|---|
| 1 | 0.00 | 90.0 | 10.0 |
| 2 | 5.00 | 90.0 | 10.0 |
| 3 | 55.00 | 40.0 | 60.0 |
| 4 | 60.00 | 40.0 | 60.0 |
| 5 | 61.00 | 90.0 | 10.0 |
| 6 | 65.00 | 90.0 | 10.0 |

MALDI-TOF analysis of Affibody A-MAL-20 kD PEG-BA-docetaxel showed a major peak at 28899 Da representing the singly charged Affibody A-MAL-20 kD PEG-BA-docetaxel; a peak at 14435 Da representing the doubly charged conjugate, and a peak at 57310 Da representing the dimerized conjugate.

The procedure above was repeated using MAL-2 kD PEG-BA-docetaxel, to yield Affibody A-MAL-2 kD PEG-BA-docetaxel. MALDI-TOF analysis of Affibody A-MAL-2 kD PEG-BA-docetaxel showed a major peak at 9845 Da representing the singly charged Affibody A-MAL-2 kD PEG-BA-docetaxel.

### Example 7

### Determination of Stability of Conjugates in Tissue Culture Growth Medium

To assess the stability of the conjugates described in Example 6 in complete tissue culture growth medium at physiological pH (pH=7.4), and pH 6.5, the conjugates were diluted to a final concentration of 25 μM in complete growth medium (RPMI-1640 containing 2 mM glutamine and 10% fetal bovine serum) pH to 7.4 and pH 6.5 (100 μL of HCl was added to 250 mL of the same complete growth medium to bring pH to 6.5). Each test condition was done in duplicate in a low protein binding 96-well block. Zero hour time point samples were immediately harvested and processed. Remaining samples were incubated in a 37° C., 5% CO² incubator for 48 and 72 hrs, and harvested at each time point.

At 0, 48 and 72 hr time points, 3 μL 2:1 PMSF (50 mM in DMSO)/Glacial Acetic Acid was added to 100 μL of media containing 25 μM of test articles and then placed in a −80° C. freezer for storage.

To analyze the samples, the samples were thawed. To the thawed samples, 100 μL of sample processing buffer (1:2, 1M Potassium Phosphate Monobasic/1M Potassium Phosphate Dibasic) and 20 μL of paclitaxel internal standard (1.25 ng/mL in DMSO) were added and mixed by vortexing. 150 μL of each sample was transferred into separate 15 mL conical tubes containing 4 mL of methyl tert-butylether, capped, and vortexed for 5-10 seconds, centrifuged at 4000 RPM for 5 minutes at 4° C. and flash frozen in a −80° C. freezer for 15 minutes. The supernatant was decanted into clean 15 mL conical tubes and evaporated to dryness under a stream of nitrogen gas using a TurboVap set at 50° C. Samples were then reconstituted with a 45:55 mixture of 10 mM Ammonium Acetate (+0.1% formic acid)/Acetonitrile, vortexed, and transferred into a 96-well plate for LC/MS/MS analysis. The results are reported in Table 4.

TABLE 4
Table of Free Docetaxel Concentrations (μM) and % Release

| | | Free Docetaxel | | Free Docetaxel | |
|---|---|---|---|---|---|
| | | Concentration | | Release | |
| | Incubation | (μM) | | (%) | |
| Compound | Time (hrs) | pH 6.5 | pH 7.4 | pH 6.5 | pH 7.4 |
| Affibody A-MAL-2kD | 0 | 0.026 | 0.029 | 0.1% | 0.1% |
| PEG-Docetaxel | 48 | 2.72 | 3.915 | 10.9% | 15.7% |
| | 72 | 3.58 | 4.425 | 14.3% | 17.7% |
| Affibody A-MAL- | 0 | 0.017 | 0.016 | 0.1% | 0.1% |
| 20kD PEG-Docetaxel | 48 | 0.682 | 1.155 | 2.7% | 4.6% |
| | 72 | 0.850 | 1.565 | 3.4% | 6.3% |

### Example 8

### Plasma Stability Testing

The rate of release of docetaxel from Affibody A-MAL-2 kD PEG-docetaxel (Example 6) and Affibody A-MAL-20 kD PEG-docetaxel (Example 6) was studied in mouse plasma and in pH 7.4 100 mM potassium phosphate buffer.

Affibody A-MAL-2 kD PEG-docetaxel (Example 6) and Affibody A-MAL-20 kD PEG-docetaxel (Example 6) were provided at stock concentrations of 1880 and 1840 μM respectively in solution with 10 mM sodium acetate and 150 sodium chloride at a pH of 4.0.

Mouse plasma and potassium phosphate buffer were pre-incubated for one hour in a 5% CO₂ water saturated atmosphere at a temperature of 37° C. In triplicate, the test article stock solutions were spiked into wells of a 96-well plate containing the pre-incubated plasma or buffer to give a final test article concentration of 5 μM and a final volume of 1 mL. The 96-well plate was then vortexed for 10 seconds and then placed in an incubator with a 5% CO₂ water saturated atmosphere set at a temperature of 37° C. At 5, 15, 30 minute and 1, 2, 4, 6 and 24 hour time points, 100 μL of solution from each incubation well was quenched by withdrawal and placement into a separate 96-well plate containing 3 μL of (2:1) phenylmethanesulfonyl fluoride (50 mM in dimethyl sulfoxide)/glacial acetic acid and then vortexed for 10 seconds.

For zero minute samples, the mouse plasma and potassium phosphate buffer were pre-treated with the (2:1) phenylmethanesulfonyl fluoride (50 mM in dimethyl sulfoxide)/glacial acetic acid quench solution prior to the addition of test articles. Upon test article addition to give a final test article concentration of 5 μM and a final volume of 1 mL the samples were vortexed for 10 seconds to mix.

All samples were processed by Liquid-Liquid-Extraction and then submitted for LC/MS/MS analysis. Rate of release of docetaxel was determined by linear regression and reported in Table 5.

TABLE 5

| | Docetaxel Release Rates | | |
|---|---|---|---|
| | | Fresh | Potassium |
| | Frozen Mouse | Mouse | Phosphate |
| Test Article | Plasma | Plasma | Buffer |
| Compound Name | ng/mL/min | ng/mL/min | ng/mL/min |
| Affibody A-MAL-2kD | 8.46 ± 0.559 | 2.45 ± 0.07 | 1.02 ± 0.03 |
| PEG-docetaxel | | | |
| Affibody A-MAL-20kD | 7.19 ± 0.669 | 2.72 ± .08 | 0.23 ± 0.006 |
| PEG-docetaxel | | | |

### Example 9

### In Vitro Cytotoxic Activity

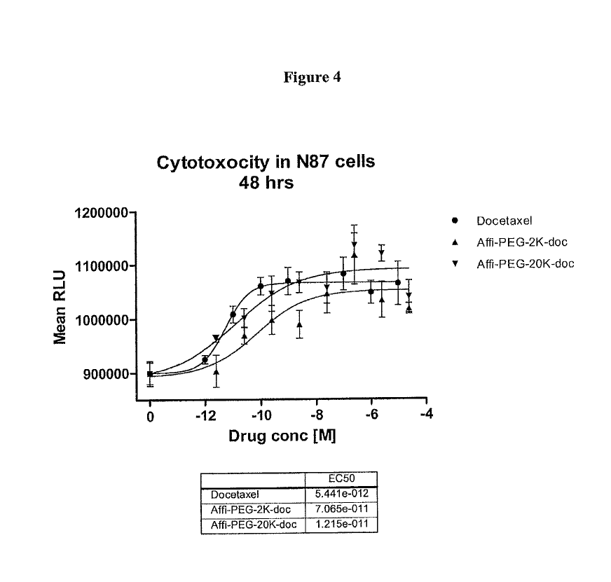

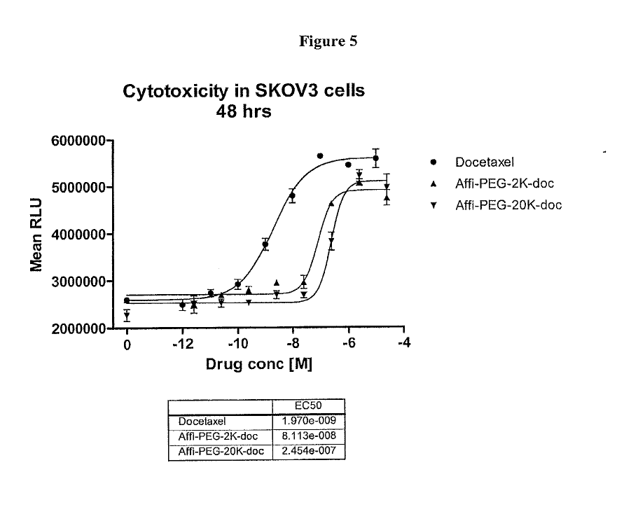

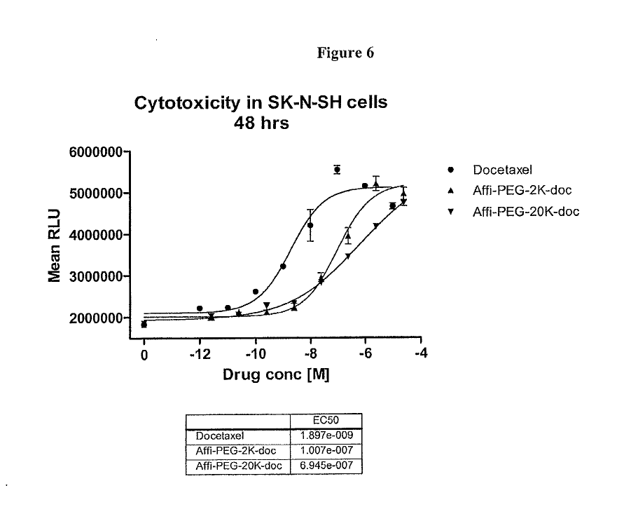

To assess cytotoxic activity of the conjugates of Example 7 (Affibody A-MAL-2 kD PEG-docetaxel and Affibody A-MAL-20 kD PEG-docetaxel) cytotoxicity assays were performed in two types of Her2 positive cell lines, NCI-N87, and SKOV-3. The conjugates were also tested in a Her2 negative cell line, SK-N-SK as an experimental control. Serial dilutions of the conjugates and control compound were prepared. Cells were lifted from the flask using 2 mL of trypsin for 5 min, neutralized with 8 mL of complete growth medium, and centrifuged at 1200 rpm for 5 min. The supernatant was aspirated and the cell pellet was washed with PBS, centrifuged, then resuspended in complete growth medium (McCoy's for SKOV3 and RPMI-1640 for N87 and SK-N-SH, supplemented with 10% heat inactivated FBS), pH 6.5. Cells were counted, diluted, and then plated in white, clear bottom 96-well plates at a density of 20,000 cells/well, then followed immediately by addition of conjugates and control compound. Cells were incubated with the test articles for 48 hours in a 37° C., 5% CO² incubator, and then analyzed for cytotoxic activity using Promega CytoTox-Glo chemiluminescent reagent (Cat#G9291). Signal was quantified with a Perkin Elmer TopCount plate reader, and the data was analyzed using the GraphPad Prism software. EC50 values for each cell line are reported in Table 6. FIGS. 4, 5, and 6 are linear regression graphs for each cell line where mean RLU is plotted vs. concentration.

TABLE 6

| | | | SK-N-SH |
|---|---|---|---|
| Test article | N87 EC50(M) | SKOV3 EC50(M) | EC50(M) |
| Docetaxel | 5.44E−12 | 1.90E−09 | 1.97E−09 |
| Affibody A-MAL- | 7.07E−11 | 1.01E−07 | 8.11E−08 |
| 2kDPEG-docetaxel | | | |
| Affibody A-MAL-20kD | 1.22E−11 | 6.95E−07 | 2.45E−07 |
| PEG-docetaxel | | | |

### Example 10

### Pharmacokinetic Profiles of IRDye800-Labeled PEG-Affibody a Conjugates in Rats

Male Sprague-Dawley rats (300-325 g) were obtained from Harlan Laboratory and housed in testing facility for 3 days for acclimation. Testing articles (IRDye800-Affibody A, 2 kD PEG-Affibody A-IRDye800, IRDye800-20 kD PEG-Affibody A IRDye800 and IRDye800-(10 kD PEG)₂-Affibody A-IRDye800, wherein the IR dye is attached to the Affibody molecule according to the procedures provided by the dye manufacturer Li-Cor Biosciences (Lincoln, Nebr.)) were diluted with 0.9% NaCl and dosed at 10 nmol/kg. The testing compounds were administered IV through jugular vein catheter. At different time points after dosing, blood samples were collected through carotid artery catheter into collection tubes containing Lithium Heparin. The blood samples were centrifuged at 10000 rpm for 10 minutes under 4 C, and the plasma supernatant was separated and stored in −65 C freezer.

At the time of sample analysis, frozen plasma samples were thawed and 25 ul of plasma for each time point was pipette into 96-well plate. Additional 100 ul of dilution buffer was added to each well. The plate was scanned using Li-Cor Odyssey scanner and the near IR fluorescence signal in 800 nm channel was obtained. Concentration standard curve was established by spiking different amount of conjugate into normal rat plasma and obtaining their near IR signal data. The drug concentration in rat plasma was obtained by comparing with the standard curve.

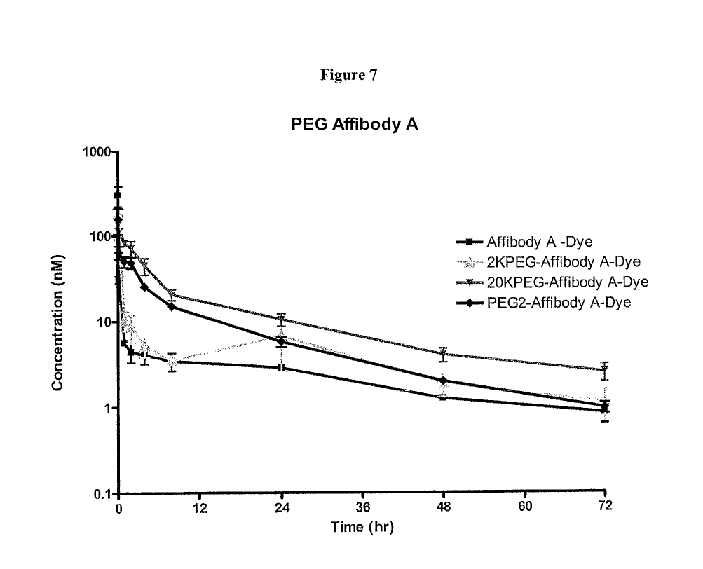

The rat PK profile was analyzed by 2-Compartment Model using WinNonLin Phoenix 6.2.1 software from Pharsight (Mountain View, Calif.). The 2-compartment IV-bolus model has 1^{st} order Elimination Rate, no lag time and used the formula: C(T)=A*exp(−Alpha*T)+B*exp(−Beta*T) to describe the bi-phase PK profiles for Affibody conjugates. Parameters A, B, Alpha, Beta, half-lives for Alpha and Beta phase (Alpha HL and Beta HL) and PK parameters such as area under the curve (AUC), clearance (CL), volume of distribution (V) and mean resident time (RMT) were calculated and reported. FIG. 7 is a plot of the Concentration vs. time for IRDye800-Affibody A, 2 kD PEG-Affibody A-IRDye800, IRDye800-20 kD PEG-Affibody A IRDye800 and IRDye800-(10 kD PEG)₂-Affibody A-IRDye800.

TABLE 7

| | | | 2 kD PEG- | | 20 kD PEG- | | (10 kD PEG)₂- | |
|---|---|---|---|---|---|---|---|---|
| | Affibody A | | Affibody A | | Affibody A | | Affibody A | |
| | Ave | sd | Ave | sd | Ave | sd | Ave | sd |
| A (nmol/L) | 588.52 | 376.81 | 210.91 | 12.48 | 102.53 | 22.77 | 662.96 | 919.93 |
| Alpha (1/hr) | 18.80 | 5.48 | 5.02 | 0.22 | 0.53 | 0.29 | 31.26 | 37.17 |
| Alpha_HL (hr) | 0.039 | 0.012 | 0.138 | 0.006 | 1.816 | 1.395 | 0.765 | 1.288 |
| B (nmol/L) | 4.96 | 0.84 | 7.68 | 2.74 | 24.43 | 13.77 | 52.96 | 32.20 |
| Beta (1/hr) | 0.03 | 0.01 | 0.03 | 0.01 | 0.04 | 0.02 | 0.15 | 0.08 |
| Beta_HL (hr) | 26.380 | 7.519 | 27.400 | 11.453 | 25.192 | 18.819 | 6.532 | 5.210 |
| MRT (hr) | 33.15 | 11.38 | 34.68 | 17.33 | 23.59 | 10.68 | 7.99 | 5.45 |
| Vss (L) | 0.46 | 0.13 | 0.30 | 0.05 | 0.07 | 0.01 | 0.06 | 0.04 |
| CL (L/hr) | 0.014 | 0.001 | 0.010 | 0.007 | 0.003 | 0.001 | 0.008 | 0.001 |
| AUC | 212.09 | 19.94 | 368.46 | 239.75 | 932.33 | 243.71 | 390.44 | 31.94 |
| (hr * nmol/L) | | | | | | | | |

### Example 11

### The Antitumor Activity of HER2 (c-erbB2) Targeted PEGylated Docetaxel in N87 (HER2 Overexpressing Gastric Carcinoma) Xenografts in Female SCID Mice

The objective of this study was to evaluate the antitumor activity of a single administration of anti-HER2 targeted PEG-conjugated docetaxel on N87 gastric carcinoma xenografts in female SCID mice.

Animal Husbandry:

Female C.B.-17 SCID mice (5-6 weeks old), purchased from Charles River Laboratories were used for this study. Upon arrival, the mice were housed in ventilated polycarbonate cages with sterile bedding at five animals per cage and acclimated for a minimum of three days. The cages were identified by cage labels or tags. Sterilized (autoclaved or irradiated) water and commercial rodent diet were supplied ad libitum. Cages, bedding and water bottles were changed at least once a week and food was replenished as needed. The animals were maintained at a temperature of 65-72° F. and a relative humidity of 30-70% in a room with a 12 hour light/dark cycle.

Tumor Cell Preparation:

All handling and preparation of cells were conducted in a biosafety cabinet for sterility and safety. N87, gastric carcinoma cells from the NEKTAR liquid nitrogen freezers were used for this study. The cells were grown in sterile tissue culture flasks with RPMI 1640 media containing 10% Fetal Bovine Serum (FBS) until 85% confluence. To prepare for implantation, the cells in the flasks were rinsed briefly with an appropriate amount of 0.25% trypsin solution. The rinsate was removed and replaced with 2 to 3 mL of fresh 0.25% trypsin solution. The flasks were then placed in a 37′C incubator for 5 minutes. When the cells dissociated, an appropriate amount of complete growth media was added to neutralize the trypsin. The suspension was centrifuged at 1000 rpm for 10 minutes after which the supernatant was discarded. The cells were washed by resuspending the pellet in an appropriate volume of serum free media. A sample was removed for counting and the rest of the suspension was again centrifuged at 1000 rpm for 10 minutes. After centrifugation, the supernatant was discarded and the cell pellet resuspended in serum free media at an appropriate volume determined by the cell count.

Tumor Cell Implantation:

All handling of animals and procedures were done in a biosafety cabinet. The animals' flanks were shaved prior to the day of implantation. The mice were anesthetized via isoflurane inhalation, when in proper anesthetic plane; the flanks were wiped with 70% isopropanol followed by implantation of the NCI N87 cells at 1×10⁷ cells/site in 0.2 mL volume (in 50% Matrigel) injected subcutaneous on the right flank of the animals. The animals were distributed accordingly based on the randomization generated by the StudyLog® software.

Treatment Groups:

There were 5 groups with 8 to 10 animals each. Included were vehicle (Saline), Docetaxel at 10 mg/kg and 20 mg/kg, Affibody A-linear 2 kD PEG-docetaxel 10 mg/kg (Example 6) (Docetaxel concentration) and Affibody A-linear 20 kD PEG-docetaxel at 10 mg/kg (Example 6) (Docetaxel concentration) respectively.

Treatment Administration:

The animals were distributed based on the randomization generated by the StudyLog® software. The mean tumor volumes on treatment day (Day 0) ranged from 209±8 mm³ to 239±15 mm³ (Mean±SEM). All test articles were diluted in sterile saline to the appropriate concentration and were administered as single dose to the appropriate treatment group via intravenous injection (i.v.). Animals were warmed in clear acrylic hotboxes to facilitate venous dilation. While in the hotboxes, the animals were closely observed for signs of overheating and distress. When the animals were amply warmed, they were removed from the hotboxes and transferred into appropriate mouse restrainers. The tails were wiped with alcohol pads and the test article in a syringe with a 27 gauge needle was injected into the lateral tail vein. Maximum treatment volume for the i.v. injections will not exceed 20 mL/kg.

Tumor Measurements and Body Weights:

Tumors size was monitored at least twice weekly. Dimensions (length and the width) were measured using digital calipers. Tumor volume was computed using the formula:

Volume=L×W²/2.

Where: L=length of the tumor in millimeters and W=width of the tumor in millimeters

Tumor growth on each animal was monitored to a maximum volume of 2000 mm². Animals were weighed prior to implantation and at least twice weekly for the duration of the study.

Results:

The smallest and largest tumor volumes for each group, as well as tumor volumes on treatment day and study termination are summarized in Table 8. Tumors with final volumes that are greater than its initial volume but less than that of the mean final volume of the vehicle control group were classified to be in a state of inhibition in Table 8.

INHIBITION: ^{Initial Vol}Treated Tumor<^{Final Vol}Treated Tumor<mean^{Final Vol}Vehicle Control

Percent Inhibition was determined by using the formula below prior to calculation of the group means:

=[1−(^{Tumor Growth}Treated Tumor/^{Mean Tumor Growth}Solvent Control]×100

Where:

^{Tumor Growth}Treated Tumor=^{Final Vol}Treated Tumor−^{Initial Vol}Treated Tumor

^{Mean Tumor Growth}Vehicle Control=^{Mean Final Volume}Vehicle Control−^{Mean Initial Volume}Vehicle Control

Tumors with final volumes that are less than its initial but greater than zero (0) were considered to be in partial regression.

REGRESSION: No Tumor (0)<^{Final Vol}Treated Tumor<^{Initial Vol}Treated Tumor

The percent regression values were calculated individually for each animal using the formula below prior to computation of the group means:

=100−[(^{Final Vol}Treated Tumor/^{Initial Vol}Treated Tumor)×100]

A combination of complete inhibition (100% inhibition) and the absence of regression (0% regression) usually characterize a state of a “stable disease”.

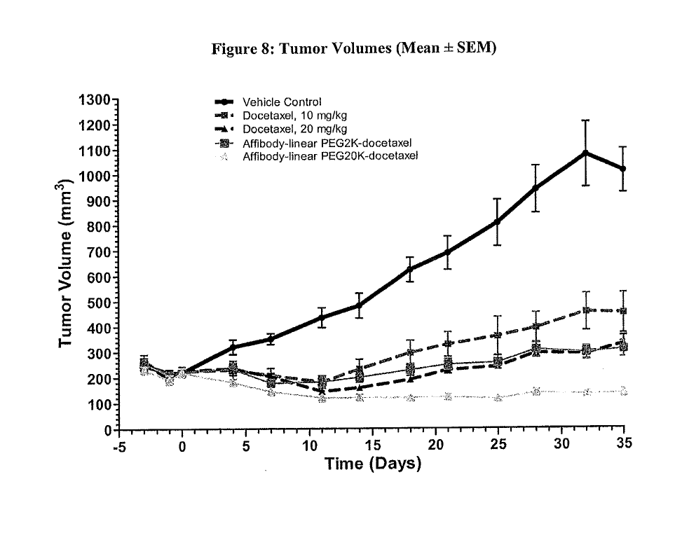

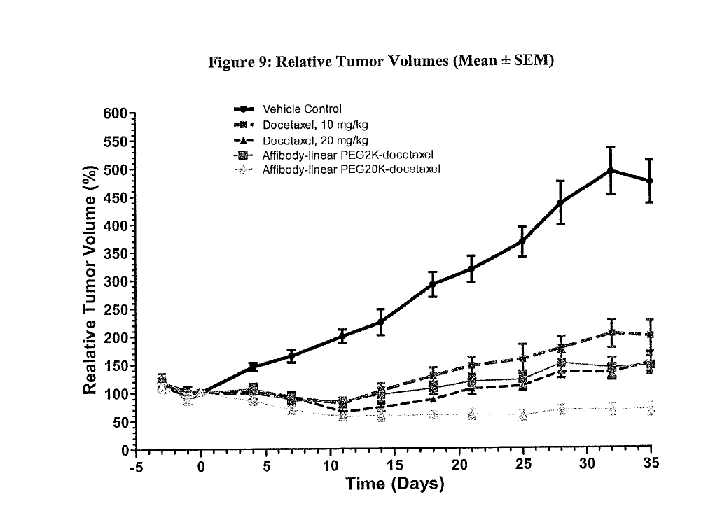

Mean tumor volumes (in mm³) were monitored 2 to 3 times a week for the duration of the study (35 days) and are presented in FIG. 8. The corresponding relative tumor values, standardized values calculated against individual tumor volumes at the start of the study and presented as percent growth are summarized in FIG. 9. Relative tumor values facilitate direct comparison of the growth curves by adjusting their origin to a single common starting point.

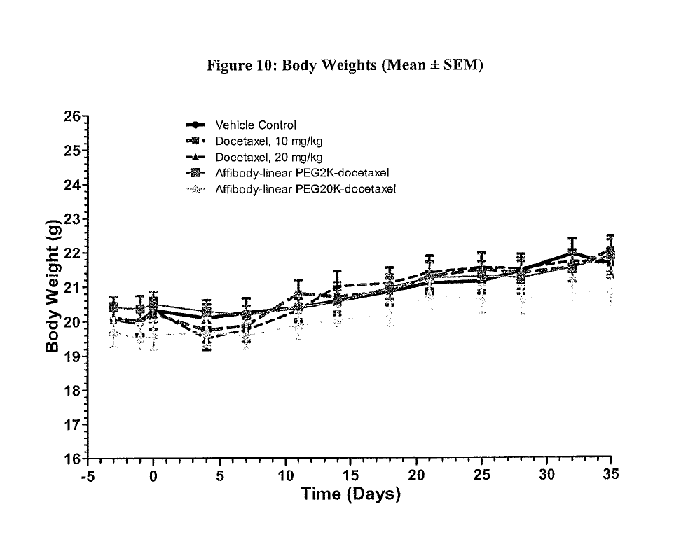

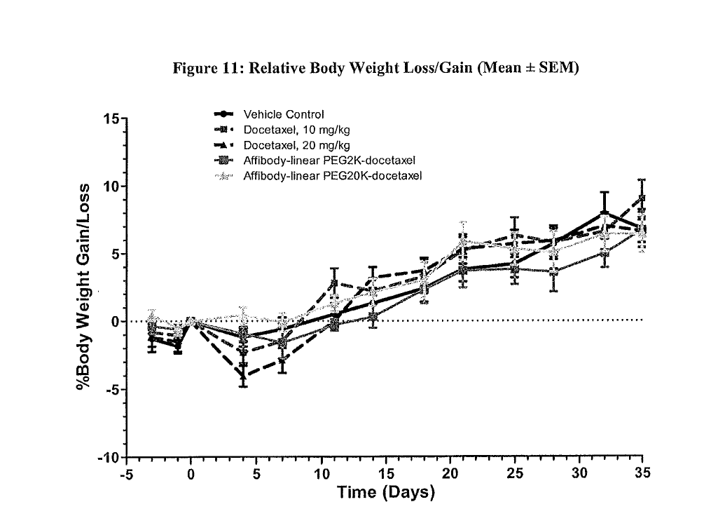

Mean body weights ranged from 19.6±0.4 g to 20.7±0.5 g (Mean±SEM) on treatment day (FIG. 10). Greatest mean body weight loss observed was 4% in group 3 (docetaxel, 20 mg.kg) on day 4 followed by group 2 (Docetaxel, 10 mg/kg) at 2% observed on days 4 and 7 (FIG. 11). No abnormal clinical observations were noted throughout the study.

Analysis of mean tumor volumes on Day 35 (study termination) using one-way ANOVA with Tukey's multiple comparison post test was performed using GraphPad Prism® (Ver. 5.04). Mean tumor volumes of all the treatment groups demonstrated significant difference from the vehicle control group. A significant difference was observed between the 20 kD PEG conjugated docetaxel group versus free docetaxel at 10 mg/kg. The results are summarized in Table 8 below.

TABLE 8

| | | Tumor | | | Tumor | Mean | |
|---|---|---|---|---|---|---|---|
| | | Volume on | Lowest | Highest | Volume on | Tumor | |
| | Docetaxel | Treatment | Tumor | Tumor | Study | Growth | |
| | Dose | Initiation | Volume | Volume | Termination | Inhibition | Regression |
| Treatment | (mg/kg) | (Day 0) | Recorded | Recorded | (Day 35) | (%) | (%) |
| Vehicle | NA | 218 ± 15 | 218 ± 15 | 1076 ± 128 | 1013 ± 86 | NA | NA |
| Control | | | (Day 0) | (Day 32) | | | |
| Docetaxel | 10 | 224 ± 19 | 182 ± 25 | 452 ± 80 | 452 ± 80 | 67.5 | 26 |
| | | | (Day 11) | (Day 35) | | (9/10) | (1/10) |
| Docetaxel | 20 | 225 ± 12 | 145 ± 8 | 330 ± 33 | 330 ± 33 | 85 | 10 |
| | | | (Day 11) | (Day 35) | | (9/10) | (1/10) |
| Affibody A- | 10 | 221 ± 21 | 183 ± 17 | 221 ± 21 | 309 ± 31 | 88 | 5 |
| linear 2 kD | | | (Day 11) | (Day 0) | | (9/10) | (1/10) |
| PEG- | | | | | | | |
| docetaxel | | | | | | | |
| Affibody A- | 10 | 219 ± 18 | 115 ± 12 | 219 ± 18 | 137 ± 16 | 88 | 44 |
| linear 20 kD | | | (Day 25) | (Day 0) | | (1/10) | (9/10) |
| PEG- | | | | | | | |
| docetaxel | | | | | | | |

Modifications and variations in the subject matter set forth in the above illustrative examples are expected to occur to those skilled in the art. Only such limitations as appear in the appended claims should be placed on any claimed invention.

1. (canceled)

2. A compound, according to the formula:
Ab-X¹-POLY-X²-Dr Formula I
wherein:
Ab is a binding moiety;
X¹ is a first spacer moiety;
POLY is a water-soluble, non-peptidic polymer;
X² is a second spacer moiety; and
Dr is a pharmacologically active agent.

3. A compound according to the formula:
[Ab-X¹-POLY]ₐ-Q¹-R-Q²-[POLY-X²-Dr]_{b} Formula II
wherein:
Ab is a binding moiety;
X¹ is a first spacer moiety;
POLY, for each occurrence, is a water-soluble, non-peptidic polymer;
X² is a second spacer moiety;
Dr is a pharmacologically active agent;
R is a residue of polyol, polythiol or polyamine bearing at from 3 to about 50 hydroxyl, thiol or amino groups;
Q¹ and Q² for each occurrence is a linker
(a) is an integer from 1 to 49; and
(b) is an integer from 1 to 49;
provided that (a)+(b) is not less than 4 and not greater than 50.

4. The compound of claim 2, wherein Ab is an Affibody® moiety.

5. The compound of claim 3, wherein the Affibody® moiety is a HER2 binding Affibody®.

6. (canceled)

7. The compound of claim 2, wherein X² is physiologically cleavable.

8. The compound of claim 2, wherein X¹ is not physiologically cleavable.

9. (canceled)

10. The compound of claim 2 wherein Dr is selected from a pharmacologically active agent useful for the treatment of breast cancer, ovarian cancer, pancreatic cancer, stomach cancer, salivary cancer, lung cancer, and esophageal cancer.

11. The compound of claim 2 wherein Dr is selected from docetaxel, topotecan, paclitaxel, anthracylcin, DM1, gemcitabine, and lapatinib.

12. The compound of claim 2, wherein the compound is selected from a compound of the formula

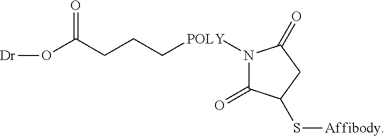

13. (canceled)

14. The compound of claim 2, wherein POLY is a poly(alkylene oxide).

15. The compound of claim 2, wherein, POLY is poly(ethylene glycol).

16. The compound of claim 2, wherein the weight-average molecular weight of the water-soluble non-peptidic polymer in the compound is from about 100 Daltons to about 150,000 Daltons.

17. The compound of claim 2, wherein the weight-average molecular weight of the water-soluble non-peptidic polymer in the compound is from about 2,000 to about 25,000.

18. The compound of claim 2, wherein the weight-average molecular weight of the water-soluble non-peptidic polymer in the compound is from about 4,000 to about 20,000.

19. The compound of claim 2, wherein the compound is selected from a compound of the formula 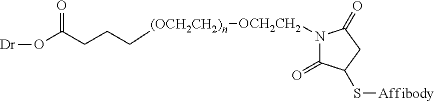
n is an integer from about 2 to about 4000.

20. The compound of claim 19, wherein n is an integer from about 30 to about 500.

21. The compound of claim 20, wherein n is an integer from about 45 to about 460.

22. (canceled)

23. A pharmaceutical composition comprising a compound of any one claim 2 and a pharmaceutically acceptable excipient.

24. (canceled)

25. (canceled)

26. A method of treating cancer in a patient comprising administering a therapeutically effective amount of a compound of claim 2 a patient in need thereof.

27. (canceled)

28. (canceled)
